SEQUENCE LISTING (136 sequences)

SEQ ID NO: 1 - protein, 5 aa, Artificial Sequence
  Synthetic Construct
RRRRR

SEQ ID NO: 2 - protein, 5 aa, Artificial Sequence
  Synthetic Construct
AAAAA

SEQ ID NO: 3 - protein, 4 aa, Artificial Sequence
  Synthetic Construct
FFFF

SEQ ID NO: 4 - protein, 5 aa, Artificial Sequence
  Synthetic Construct
  MOD_RES (3)..(3): phosphocoumaryl amino propionic acid
DEXLI

SEQ ID NO: 5 - protein, 7 aa, Artificial Sequence
  Synthetic Construct
AAAAAAA

SEQ ID NO: 6 - protein, 5 aa, Artificial Sequence
  Synthetic Construct
RARAR

SEQ ID NO: 7 - protein, 5 aa, Artificial Sequence
  Synthetic Construct
DADAD

SEQ ID NO: 8 - protein, 4 aa, Artificial Sequence
  Synthetic Construct
  MOD_RES (2)..(2): norleucine
  MOD_RES (3)..(3): 2-aminobutyric acid
DXXD

SEQ ID NO: 9 - protein, 4 aa, Artificial Sequence
  Synthetic Construct
  misc_feature (1)..(1): Xaa can be any naturally occurring amino acid
  MOD_RES (2)..(2): 2-aminobutyric acid
XTRV

SEQ ID NO: 10 - protein, 7 aa, Artificial Sequence
  Synthetic Construct
  MOD_RES (2)..(2): 2  L-2-naphthylalanine
FXRRRRQ

SEQ ID NO: 11 - protein, 5 aa, Artificial Sequence
  Synthetic Construct
  MOD_RES (2)..(2): L-homoproline
  MOD_RES (4)..(4): L-4-(phosphonodifluoromethyl)phenylalanine
PXGXY

SEQ ID NO: 12 - protein, 5 aa, Artificial Sequence
  Synthetic Construct
  MOD_RES (2)..(2): L-homoproline
  MOD_RES (4)..(5): L-4-(phosphonodifluoromethyl)phenylalanine
SXIXX

SEQ ID NO: 13 - protein, 5 aa, Artificial Sequence
  Synthetic Construct
  MOD_RES (4)..(4): L-4-(phosphonodifluoromethyl)phenylalanine
IHIXI

SEQ ID NO: 14 - protein, 1 aa, Artificial Sequence
  Synthetic Construct
  MOD_RES (1)..(1): D-amino acid
X

SEQ ID NO: 15 - protein, 1 aa, Artificial Sequence
  Synthetic Construct
  MOD_RES (1)..(1): D-amino acid
X

SEQ ID NO: 16 - protein, 1 aa, Artificial Sequence
  Synthetic Construct
  MOD_RES (1)..(1): D-amino acid
X

SEQ ID NO: 17 - protein, 5 aa, Artificial Sequence
  Synthetic Construct
  MOD_RES (2)..(2): L-phenylglycine
  MOD_RES (4)..(4): L-4-(phosphonodifluoromethyl)phenylalanine
TXAXG

SEQ ID NO: 18 - protein, 1 aa, Artificial Sequence
  Synthetic Construct
  MOD_RES (1)..(1): D-amino acid
X

SEQ ID NO: 19 - protein, 1 aa, Artificial Sequence
  Synthetic Construct
  MOD_RES (1)..(1): D-amino acid
X

SEQ ID NO: 20 - protein, 1 aa, Artificial Sequence
  Synthetic Construct
  MOD_RES (1)..(1): D-amino acid
X

SEQ ID NO: 21 - protein, 5 aa, Artificial Sequence
  Synthetic Construct
  MOD_RES (4)..(4): L-4-(phosphonodifluoromethyl)phenylalanine
SPGXH

SEQ ID NO: 22 - protein, 5 aa, Artificial Sequence
  Synthetic Construct
  MOD_RES (1)..(1): L-homoproline
  MOD_RES (4)..(4): L-4-(phosphonodifluoromethyl)phenylalanine
XYIXH

SEQ ID NO: 23 - protein, 1 aa, Artificial Sequence
  Synthetic Construct
  MOD_RES (1)..(1): D-amino acid
X

SEQ ID NO: 24 - protein, 1 aa, Artificial Sequence
  Synthetic Construct
  MOD_RES (1)..(1): D-amino acid
X

SEQ ID NO: 25 - protein, 6 aa, Artificial Sequence
  Synthetic Construct
  MOD_RES (1)..(1): L-4-fluorophenylalanine
  MOD_RES (4)..(4): L-4-(phosphonodifluoromethyl)phenylalanine
XSIXQF

SEQ ID NO: 26 - protein, 1 aa, Artificial Sequence
  Synthetic Construct
  MOD_RES (1)..(1): D-amino acid
X

SEQ ID NO: 27 - protein, 1 aa, Artificial Sequence
  Synthetic Construct
  MOD_RES (1)..(1): D-amino acid
X

SEQ ID NO: 28 - protein, 5 aa, Artificial Sequence
  Synthetic Construct
  MOD_RES (3)..(3): L-phenylglycine
  MOD_RES (4)..(4): L-4-(phosphonodifluoromethyl)phenylalanine
  MOD_RES (5)..(5): L-norleucine
IPXXX

SEQ ID NO: 29 - protein, 5 aa, Artificial Sequence
  Synthetic Construct
  MOD_RES (2)..(2): L-homoproline
  MOD_RES (3)..(3): L-4-fluorophenylalanine
  MOD_RES (4)..(4): L-4-(phosphonodifluoromethyl)phenylalanine
  MOD_RES (5)..(5): L-homoproline
QXXXX

SEQ ID NO: 30 - protein, 1 aa, Artificial Sequence
  Synthetic Construct
  MOD_RES (1)..(1): D-amino acid
X

SEQ ID NO: 31 - protein, 1 aa, Artificial Sequence
  Synthetic Construct
  MOD_RES (1)..(1): D-amino acid
X

SEQ ID NO: 32 - protein, 1 aa, Artificial Sequence
  Synthetic Construct
  MOD_RES (1)..(1): D-amino acid
X

SEQ ID NO: 33 - protein, 1 aa, Artificial Sequence
  Synthetic Construct
  MOD_RES (1)..(1): D-amino acid
X

SEQ ID NO: 34 - protein, 1 aa, Artificial Sequence
  Synthetic Construct
  MOD_RES (1)..(1): D-amino acid
X

SEQ ID NO: 35 - protein, 1 aa, Artificial Sequence
  Synthetic Construct
  MOD_RES (1)..(1): D-amino acid
X

SEQ ID NO: 36 - protein, 1 aa, Artificial Sequence
  Synthetic Construct
  MOD_RES (1)..(1): D-amino acid
X

SEQ ID NO: 37 - protein, 1 aa, Artificial Sequence
  Synthetic Construct
  MOD_RES (1)..(1): D-amino acid
X

SEQ ID NO: 38 - protein, 1 aa, Artificial Sequence
  Synthetic Construct
  MOD_RES (1)..(1): D-amino acid
X

SEQ ID NO: 39 - protein, 1 aa, Artificial Sequence
  Synthetic Construct
  MOD_RES (1)..(1): D-amino acid
X

SEQ ID NO: 40 - protein, 1 aa, Artificial Sequence
  Synthetic Construct
  MOD_RES (1)..(1): D-amino acid
X

SEQ ID NO: 41 - protein, 1 aa, Artificial Sequence
  Synthetic Construct
  MOD_RES (1)..(1): D-amino acid
X

SEQ ID NO: 42 - protein, 1 aa, Artificial Sequence
  Synthetic Construct
  MOD_RES (1)..(1): D-amino acid
X

SEQ ID NO: 43 - protein, 1 aa, Artificial Sequence
  Synthetic Construct
  MOD_RES (1)..(1): D-amino acid
X

SEQ ID NO: 44 - protein, 1 aa, Artificial Sequence
  Synthetic Construct
  MOD_RES (1)..(1): D-amino acid
X

SEQ ID NO: 45 - protein, 1 aa, Artificial Sequence
  Synthetic Construct
  MOD_RES (1)..(1): D-amino acid
X

SEQ ID NO: 46 - protein, 1 aa, Artificial Sequence
  Synthetic Construct
  MOD_RES (1)..(1): D-amino acid
X

SEQ ID NO: 47 - protein, 5 aa, Artificial Sequence
  Synthetic Construct
  MOD_RES (2)..(2): L-phenylglycine
  MOD_RES (4)..(4): L-4-(phosphonodifluoromethyl)phenylalanine
  MOD_RES (5)..(5): L-homoproline
PXHXX

SEQ ID NO: 48 - protein, 1 aa, Artificial Sequence
  Synthetic Construct
  MOD_RES (1)..(1): D-amino acid
X

SEQ ID NO: 49 - protein, 5 aa, Artificial Sequence
  Synthetic Construct
  MOD_RES (4)..(4): L-4-(phosphonodifluoromethyl)phenylalanine
PAHXG

SEQ ID NO: 50 - protein, 5 aa, Artificial Sequence
  Synthetic Construct
  MOD_RES (4)..(4): L-4-(phosphonodifluoromethyl)phenylalanine
AYHXI

SEQ ID NO: 51 - protein, 1 aa, Artificial Sequence
  Synthetic Construct
  MOD_RES (1)..(1): D-amino acid
X

SEQ ID NO: 52 - protein, 1 aa, Artificial Sequence
  Synthetic Construct
  MOD_RES (1)..(1): D-amino acid
X

SEQ ID NO: 53 - protein, 1 aa, Artificial Sequence
  Synthetic Construct
  MOD_RES (1)..(1): D-amino acid
X

SEQ ID NO: 54 - protein, 1 aa, Artificial Sequence
  Synthetic Construct
  MOD_RES (1)..(1): D-amino acid
X

SEQ ID NO: 55 - protein, 1 aa, Artificial Sequence
  Synthetic Construct
  MOD_RES (1)..(1): D-amino acid
X

SEQ ID NO: 56 - protein, 1 aa, Artificial Sequence
  Synthetic Construct
  MOD_RES (1)..(1): D-amino acid
X

SEQ ID NO: 57 - protein, 1 aa, Artificial Sequence
  Synthetic Construct
  MOD_RES (1)..(1): D-amino acid
X

SEQ ID NO: 58 - protein, 6 aa, Artificial Sequence
  Synthetic Construct
FFRRRQ

SEQ ID NO: 59 - protein, 1 aa, Artificial Sequence
  Synthetic Construct
  MOD_RES (1)..(1): D-amino acid
X

SEQ ID NO: 60 - protein, 1 aa, Artificial Sequence
  Synthetic Construct
  MOD_RES (1)..(1): D-amino acid
X

SEQ ID NO: 61 - protein, 6 aa, Artificial Sequence
  Synthetic Construct
FRFRRQ

SEQ ID NO: 62 - protein, 6 aa, Artificial Sequence
  Synthetic Construct
FRRFRQ

SEQ ID NO: 63 - protein, 6 aa, Artificial Sequence
  Synthetic Construct
FRRRFQ

SEQ ID NO: 64 - protein, 6 aa, Artificial Sequence
  Synthetic Construct
  MOD_RES (2)..(2): L-2-naphthylalanine
GXRRRQ

SEQ ID NO: 65 - protein, 6 aa, Artificial Sequence
  Synthetic Construct
FFFRAQ

SEQ ID NO: 66 - protein, 6 aa, Artificial Sequence
  Synthetic Construct
FFFRRQ

SEQ ID NO: 67 - protein, 7 aa, Artificial Sequence
  Synthetic Construct
FFRRRRQ

SEQ ID NO: 68 - protein, 7 aa, Artificial Sequence
  Synthetic Construct
FRRFRRQ

SEQ ID NO: 69 - protein, 7 aa, Artificial Sequence
  Synthetic Construct
FRRRFRQ

SEQ ID NO: 70 - protein, 7 aa, Artificial Sequence
  Synthetic Construct
RFFRRRQ

SEQ ID NO: 71 - protein, 7 aa, Artificial Sequence
  Synthetic Construct
RFRRFRQ

SEQ ID NO: 72 - protein, 7 aa, Artificial Sequence
  Synthetic Construct
FRFRRRQ

SEQ ID NO: 73 - protein, 7 aa, Artificial Sequence
  Synthetic Construct
FFFRRRQ

SEQ ID NO: 74 - protein, 7 aa, Artificial Sequence
  Synthetic Construct
FFRRRFQ

SEQ ID NO: 75 - protein, 7 aa, Artificial Sequence
  Synthetic Construct
FRFFRRQ

SEQ ID NO: 76 - protein, 7 aa, Artificial Sequence
  Synthetic Construct
RRFFFRQ

SEQ ID NO: 77 - protein, 7 aa, Artificial Sequence
  Synthetic Construct
FFRFRRQ

SEQ ID NO: 78 - protein, 7 aa, Artificial Sequence
  Synthetic Construct
FFRRFRQ

SEQ ID NO: 79 - protein, 7 aa, Artificial Sequence
  Synthetic Construct
FRRFFRQ

SEQ ID NO: 80 - protein, 7 aa, Artificial Sequence
  Synthetic Construct
FRRFRFQ

SEQ ID NO: 81 - protein, 7 aa, Artificial Sequence
  Synthetic Construct
FRFRFRQ

SEQ ID NO: 82 - protein, 7 aa, Artificial Sequence
  Synthetic Construct
RFFRFRQ

SEQ ID NO: 83 - protein, 7 aa, Artificial Sequence
  Synthetic Construct
  MOD_RES (2)..(2): L-2-naphthylalanine
GXRRRRQ

SEQ ID NO: 84 - protein, 8 aa, Artificial Sequence
  Synthetic Construct
FFFRRRRQ

SEQ ID NO: 85 - protein, 8 aa, Artificial Sequence
  Synthetic Construct
RFFRRRRQ

SEQ ID NO: 86 - protein, 8 aa, Artificial Sequence
  Synthetic Construct
RRFFRRRQ

SEQ ID NO: 87 - protein, 8 aa, Artificial Sequence
  Synthetic Construct
RFFFRRRQ

SEQ ID NO: 88 - protein, 8 aa, Artificial Sequence
  Synthetic Construct
RRFFFRRQ

SEQ ID NO: 89 - protein, 8 aa, Artificial Sequence
  Synthetic Construct
FFRRFRRQ

SEQ ID NO: 90 - protein, 8 aa, Artificial Sequence
  Synthetic Construct
FFRRRRFQ

SEQ ID NO: 91 - protein, 8 aa, Artificial Sequence
  Synthetic Construct
FRRFFRRQ

SEQ ID NO: 92 - protein, 9 aa, Artificial Sequence
  Synthetic Construct
FFFRRRRRQ

SEQ ID NO: 93 - protein, 10 aa, Artificial Sequence
  Synthetic Construct
FFFRRRRRRQ

SEQ ID NO: 94 - protein, 1 aa, Artificial Sequence
  Synthetic Construct
  MOD_RES (1)..(1): D-amino acid
X

SEQ ID NO: 95 - protein, 7 aa, Artificial Sequence
  Synthetic Construct
  MOD_RES (1)..(2): L-4-fluorophenylalanine
XXRRRRQ

SEQ ID NO: 96 - protein, 1 aa, Artificial Sequence
  Synthetic Construct
  MOD_RES (1)..(1): D-amino acid
X

SEQ ID NO: 97 - protein, 1 aa, Artificial Sequence
  Synthetic Construct
  MOD_RES (1)..(1): D-amino acid
X

SEQ ID NO: 98 - protein, 1 aa, Artificial Sequence
  Synthetic Construct
  MOD_RES (1)..(1): D-amino acid
X

SEQ ID NO: 99 - protein, 1 aa, Artificial Sequence
  Synthetic Construct
  MOD_RES (1)..(1): D-amino acid
X

SEQ ID NO: 100 - protein, 1 aa, Artificial Sequence
  Synthetic Construct
  MOD_RES (1)..(1): D-amino acid
X

SEQ ID NO: 101 - protein, 1 aa, Artificial Sequence
  Synthetic Construct
  MOD_RES (1)..(1): D-amino acid
X

SEQ ID NO: 102 - protein, 1 aa, Artificial Sequence
  Synthetic Construct
  MOD_RES (1)..(1): D-amino acid
X

SEQ ID NO: 103 - protein, 1 aa, Artificial Sequence
  Synthetic Construct
  MOD_RES (1)..(1): D-amino acid
X

SEQ ID NO: 104 - protein, 1 aa, Artificial Sequence
  Synthetic Construct
  MOD_RES (1)..(1): D-amino acid
X

SEQ ID NO: 105 - protein, 1 aa, Artificial Sequence
  Synthetic Construct
  MOD_RES (1)..(1): D-amino acid
X

SEQ ID NO: 106 - protein, 1 aa, Artificial Sequence
  Synthetic Construct
  MOD_RES (1)..(1): D-amino acid
X

SEQ ID NO: 107 - protein, 8 aa, Artificial Sequence
  Synthetic Construct
CWWRRRRC

SEQ ID NO: 108 - protein, 9 aa, Artificial Sequence
  Synthetic Construct
CWWVRRRRC

SEQ ID NO: 109 - protein, 8 aa, Artificial Sequence
  Synthetic Construct
CFWRRRRC

SEQ ID NO: 110 - protein, 8 aa, Artificial Sequence
  Synthetic Construct
CWWWRRRC

SEQ ID NO: 111 - protein, 1 aa, Artificial Sequence
  Synthetic Construct
  MOD_RES (1)..(1): D-amino acid
X

SEQ ID NO: 112 - protein, 1 aa, Artificial Sequence
  Synthetic Construct
  MOD_RES (1)..(1): D-amino acid
X

SEQ ID NO: 113 - protein, 1 aa, Artificial Sequence
  Synthetic Construct
  MOD_RES (1)..(1): D-amino acid
X

SEQ ID NO: 114 - protein, 1 aa, Artificial Sequence
  Synthetic Construct
  MOD_RES (1)..(1): D-amino acid
X

SEQ ID NO: 115 - protein, 1 aa, Artificial Sequence
  Synthetic Construct
  MOD_RES (1)..(1): D-amino acid
X

SEQ ID NO: 116 - protein, 1 aa, Artificial Sequence
  Synthetic Construct
  MOD_RES (1)..(1): D-amino acid
X

SEQ ID NO: 117 - protein, 1 aa, Artificial Sequence
  Synthetic Construct
  MOD_RES (1)..(1): D-amino acid
X

SEQ ID NO: 118 - protein, 1 aa, Artificial Sequence
  Synthetic Construct
  MOD_RES (1)..(1): D-amino acid
X

SEQ ID NO: 119 - protein, 1 aa, Artificial Sequence
  Synthetic Construct
  MOD_RES (1)..(1): D-amino acid
X

SEQ ID NO: 120 - protein, 1 aa, Artificial Sequence
  Synthetic Construct
  misc_feature (1)..(1): Xaa can be any naturally occurring amino acid
X

SEQ ID NO: 121 - protein, 1 aa, Artificial Sequence
  Synthetic Construct
  MOD_RES (1)..(1): D-amino acid
X

SEQ ID NO: 122 - protein, 1 aa, Artificial Sequence
  Synthetic Construct
  MOD_RES (1)..(1): D-amino acid
X

SEQ ID NO: 123 - protein, 1 aa, Artificial Sequence
  Synthetic Construct
  MOD_RES (1)..(1): D-amino acid
X

SEQ ID NO: 124 - protein, 8 aa, Artificial Sequence
  Synthetic Construct
  MOD_RES (2)..(2): L-2-naphthylalanine
FXRRRRRQ

SEQ ID NO: 125 - protein, 7 aa, Artificial Sequence
  Synthetic Construct
  MOD_RES (5)..(5): L-2-naphthylalanine
RRFRXRQ

SEQ ID NO: 126 - protein, 8 aa, Artificial Sequence
  Synthetic Construct
  MOD_RES (3)..(3): L-2-naphthylalanine
FFXRRRRQ

SEQ ID NO: 127 - protein, 8 aa, Artificial Sequence
  Synthetic Construct
  MOD_RES (6)..(6): L-2-naphthylalanine
RFRFRXRQ

SEQ ID NO: 128 - protein, 6 aa, Artificial Sequence
  Synthetic Construct
  MOD_RES (2)..(2): L-2-naphthylalanine
FXRRRQ

SEQ ID NO: 129 - protein, 7 aa, Artificial Sequence
  Synthetic Construct
  MOD_RES (6)..(6): L-2-naphthylalanine
FRRRRXQ

SEQ ID NO: 130 - protein, 1 aa, Artificial Sequence
  Synthetic Construct
  MOD_RES (1)..(1): D-amino acid
X

SEQ ID NO: 131 - protein, 7 aa, Artificial Sequence
  Synthetic Construct
  MOD_RES (3)..(3): L-2-naphthylalanine
RRXFRRQ

SEQ ID NO: 132 - protein, 1 aa, Artificial Sequence
  Synthetic Construct
  MOD_RES (1)..(1): D-amino acid
X

SEQ ID NO: 133 - protein, 1 aa, Artificial Sequence
  Synthetic Construct
  MOD_RES (1)..(1): D-amino acid
X

SEQ ID NO: 134 - protein, 1 aa, Artificial Sequence
  Synthetic Construct
  MOD_RES (1)..(1): D-amino acid
X

SEQ ID NO: 135 - protein, 1 aa, Artificial Sequence
  Synthetic Construct
  MOD_RES (1)..(1): D-amino acid
X

SEQ ID NO: 136 - protein, 1 aa, Artificial Sequence
  Synthetic Construct
  MOD_RES (1)..(1): D-amino acid
X

(19) United States
(12) Patent Application Publication
(10) Pub. No.: US 2019/0309020 A1
(43) Pub. Date: Oct. 10, 2019

(54) CELL-PENETRATING PEPTIDE SEQUENCES

(71) Applicant: Ohio State Innovation Foundation, Columbus, OH (US)

(72) Inventors: Dehua PEI, Columbus, OH (US); Ziqing QIAN, Columbus, OH (US)

(21) Appl. No.: 16/462,922

(22) PCT Filed: Nov. 22, 2017
(86) PCT No.: PCT/US2017/062951

§ 371 Date: May 21, 2019

Related U.S. Application Data
(60) Provisional application No. 62/425,438, filed on Nov. 22, 2016.
(60) Provisional application No. 62/425,550, filed on Nov. 22, 2016.
(60) Provisional application No. 62/438,141, filed on Dec. 22, 2016.
(60) Provisional application No. 62/507,482, filed on May 17, 2017.

(51) Int. Cl. C07K 7/06; A61K 47/64; C07K 7/64; A61K 49/00

(52) U.S. Cl. CPC C07K 7/06; A61K 47/64; A61K 38/00; A61K 49/0056; C07K 7/64

(57) ABSTRACT

Disclosed are cell-penetrating cyclic peptides. The peptides can be used to deliver peptides and other biologically active moieties into cells. Formulations containing the cell-penetrating cyclic peptides are also described.

### CROSS-REFERENCE TO RELATED APPLICATIONS

This application claims priority to U.S. App. No. 62/425,550, filed on Nov. 22, 2016, U.S. App. No. 62/425,438, filed on Nov. 22, 2016, U.S. App. No. 62/438,141, filed Dec. 22, 2016, and U.S. App. No. 62/507,483, filed on May 17, 2017, the contents of each of which are herein incorporated by reference in its entirety for all purposes.

### STATEMENT REGARDING FEDERALLY FUNDED RESEARCH

This invention was made with government support under grant numbers GM062820 and GM110208 awarded by the National Institutes of Health. The government has certain rights in this invention.

### BACKGROUND

The plasma membrane presents a major challenge in drug discovery, especially for biologics such as peptides, proteins and nucleic acids. One potential strategy to subvert the membrane barrier and deliver the biologics into cells is to attach them to “cell-penetrating peptides (CPPs)”. Despite three decades of investigation, the fundamental basis for CPP activity remains elusive. CPPs that enter cells via endocytosis must exit from endocytic vesicles in order to reach the cytosol. Unfortunately, the endosomal membrane has proven to be a significant barrier towards cytoplasmic delivery by these CPPs. What are thus needed are new cell penetrating peptides and compositions comprising such peptides that can be used to deliver agents to various cell types.

The compositions and methods disclosed herein address these and other needs.

### SUMMARY

In some embodiments, the present disclosure provides for cyclic peptides according to one of Formula I-A to I-E:

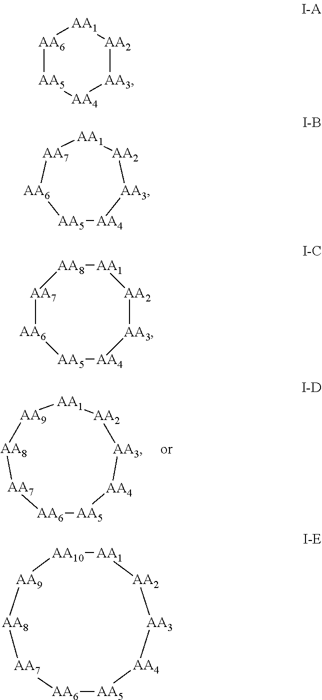

wherein each of AA₁, AA₂, AA₃, AA₄, AA₅, AA₆, AA₇, AA₈, AA₉, and AA₁₀, when present, are independently selected from an amino acid; and
wherein:
   two or three of AA₁, AA₂, AA₃, AA₄, AA₅, AA₆, AA₇, AA₈, AA₉, or AA₁₀, when present, are arginine, with the remaining amino acids thereof being an amino acid other than arginine; and
   at least two of AA₁, AA₂, AA₃, AA₄, AA₅, AA₆, AA₇, AA₈, AA₉, or AA₁₀, when present, are independently a hydrophobic amino acid.

In some embodiments, each hydrophobic amino acid is independently selected from the group consisting of phenylglycine, leucine, isoleucine, noroleucine, methionine, phenylalanine, homophenylalanine, cyclohexylalanine, tyrosine, piperidine-2-carboxylate, tryptophan, proline, 3-(3-benzothienyl)-alanine, and naphthylalanine, each of which is optionally substituted with one or more substituents. In other embodiments, each hydrophobic amino acid is independently piperidine-2-carboxylate, naphthyl alanine, tryptophan, 3-(3-benzothienyl)-alanine, or phenylalanine, each of which is optionally substituted with one or more substituents. In some embodiments, at least one hydrophobic amino acid has a hydrophobicity greater than or equal to phenylalanine.

In certain embodiments, at least two of the amino acids have the opposite chirality. In further embodiments, at least two of the amino acids have the same chirality.

In some embodiments, one arginine is adjacent to one hydrophobic amino acid. In further embodiments, the arginine has the same chirality as the adjacent hydrophobic amino acid.

In some embodiments, at least two arginines are adjacent to each other. In other embodiments, three arginines are adjacent to each other. In some embodiments, at least two hydrophobic amino acids are adjacent to each other. In other embodiments, at least three hydrophobic amino acids are adjacent to each other.

In some embodiments, any four adjacent amino acids are AA_{H2}-AA_{H1}-R-r, AA_{H2}-AA_{H1}-r-R, R-r-AA_{H1}-AA_{H2}, or r-R-AA_{H1}-AA_{H2}, wherein each of AA_{H1} and AA_{H2} are independently a hydrophobic amino acid.

Accordingly, in various embodiments, the cyclic peptides disclosed herein (e.g., the cyclic peptides according to Formula I, and I-A to I-E) have a structure according to any of Formula II-A to II-D:

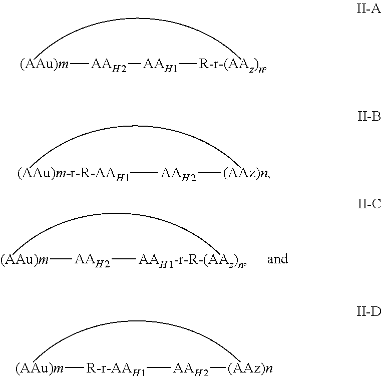

wherein:
each of AA_{H1} and AA_{H2} are independently a hydrophobic amino acid;
at each instance AAu and AAz are independently any amino acid;
with at most one of each AAu and each AAz being arginine; and

wherein:
each of m and n are independently a number from 0 to 6, provided that at least one of m or n is not 0 and the total number of amino acids is from 6 to 10.

In some embodiments, each of AA_{H1} and AA_{H2} are independently piperidine-2-carboxylate, naphthylalanine, tryptophan, 3-(3-benzothienyl)-alanine, or phenylalanine, each of which is optionally substituted with one or more substituents. In other embodiments, each of AA_{H1} and AA_{H2} are independently a hydrophobic amino acid having a hydrophobicity greater than or equal to phenylalanine (e.g., using any of the scales provided herein in Table 2). In still other embodiments, AA_{H1} is naphthylalanine. In yet still other embodiments, AA_{H1} and AA_{H2} are naphthylalanine.

In some embodiments, AA_{H1} is a hydrophobic amino acid with a side chain having a large solvent-accessible surface area (SASA). In further embodiments, the large SASA is at least about 200 Å². In still further embodiments, the large SASA is in the range of from about 200 Å² to about 1,000 Å². In other embodiments, AA_{H2} is a hydrophobic amino acid with a side chain having a SASA that is less than or equal to the SASA of the side chain of AA_{H1}. In still other embodiments, when any AA_{U} or any AA_{Z} is a hydrophobic amino acid, said hydrophobic amino acid has a side chain with SASA which is less than AA_{H1}.

In some embodiments, AA_{H1} and AA_{H2} have the same or opposite chirality. In some other embodiments, AA_{H1} and AA_{H2} have the opposite chirality. In still other embodiments, AA_{H1} has the same chirality as the adjacent arginine.

In certain embodiments, the peptides disclosed herein comprise one of the following sequences: AA_{H2}-AA_{H1}-R-r-R, AA_{H2}-AA_{H1}-R-r-r, AA_{H2}-AA_{H1}-r-R-R, AA_{H2}-AA_{H1}-r-R-r, R-R-r-AA_{H1}-AA_{H2}, r-R-r-AA_{H1}-AA_{H2}, r-r-R-AA_{H1}-AA_{H2}, or, R-r-R-AA_{H1}-AA_{H2}. In other embodiments, the peptides have one of the following sequences AA_{H2}-LAA_{H1}-R-r-R, AA_{H2}-DAA_{H1}-r-R-r, r-R-r-DAA_{H1}-AA_{H2}, or R-r-R-LAA_{H1}-AA_{H2}.

In some embodiments, the relative cytosolic delivery efficiency of the cyclic peptides disclosed herein is improved by an amount in the range of from about 50% to about 500% compared to cyclo(FΦRRRRQ) (SEQ ID NO:10). In some embodiments, the cyclic peptide has a relative cytosolic delivery efficiency which is improved by about 175% to about 250% compared to cyclo(FΦRRRRQ) (SEQ ID NO:10). In some such embodiments, the cyclic peptide comprises the following sequence: FfFRr, e.g., cyclo(FfFRrQ) (SEQ ID NO:96). In other embodiments, the cyclic peptide has a relative cytosolic delivery efficiency which is improved by about 150% to about 400% compared to cyclo(FΦRRRRQ) (SEQ ID NO:10). In some such embodiments, the cyclic peptide comprises the following sequence: fFFrRr (SEQ ID NO:132), e.g., cyclo(fFfrRrQ) (SEQ ID NO:97). In still other embodiments, the cyclic peptide has a cytosolic delivery efficiency which is improved by about 75% to about 275% compared to cyclo(FΦRRRRQ) (SEQ ID NO:10). In some such embodiments, the cyclic peptide comprises the following sequence: fFfRrR (SEQ ID NO:133), e.g., cyclo(fFfRrRQ) (SEQ ID NO:98). In yet still other embodiments, the cyclic peptide has a cytosolic delivery efficiency which is improved by about 150% to about 250% compared to cyclo(FΦRRRRQ) (SEQ ID NO:10). In some such embodiments, the cyclic peptide comprises the following sequence: FfFrRr (SEQ ID NO:134), e.g., cyclo(FfFrRrQ) (SEQ ID NO:99). In some embodiments, the cyclic peptide has a cytosolic delivery efficiency which is improved by about 200% to about 450% compared to cyclo(FΦRRRRQ) (SEQ ID NO:10). In some such comprising the following sequence: fFϕrRr (SEQ ID NO:135), e.g., cyclo(fFϕrRrQ) (SEQ ID NO:100). In other embodiments, peptide has a cytosolic delivery efficiency which is improved by about 250% to about 450% compared to cyclo(FΦRRRRQ) (SEQ ID NO:10). In some such embodiments, the cyclic peptide comprises the following sequence: fΦfrRr (SEQ ID NO:136), e.g., cyclo(fΦfrRrQ) (SEQ ID NO:101).

Also disclosed herein are cyclic peptides according to Formula III-A to III-D:

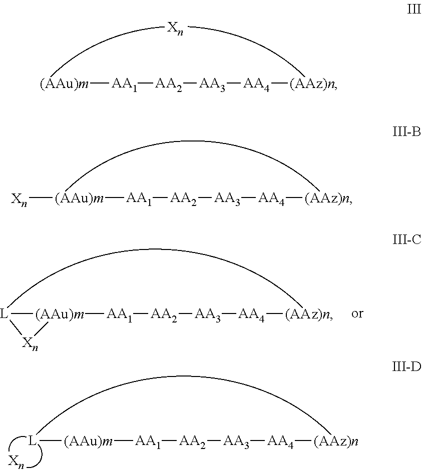

wherein:
each of AA₁, AA₂, AA₃, and AA₄, are independently selected from an amino acid;
each of AA_{U} and AA_{Z}, at each instance, are independently selected from an amino acid;
each of m and n are a number from 0 to 6, provided that at least one of m or n is not 0;
Xₙ is a cargo moiety thereof;
L is a linker moiety;

wherein:
two or three of AA₁, AA₂, AA₃, AA₄, each AA_{U}, and each AA_{Z} are arginine, with the remaining amino acids thereof being an amino acid other than arginine;
at least two of AA₁, AA₂, AA₃, AA₄, each AA_{U}, and each AA_{Z} are independently a hydrophobic amino acid; and
wherein when Xₙ is attached to AA_{U}, m is not 0.

In some embodiments, each hydrophobic amino acid is independently selected from the group consisting of phenylglycine, leucine, isoleucine, noroleucine, methionine, phenylalanine, homophenylalanine, cyclohexylalanine, tyrosine, piperidine-2-carboxylate, tryptophan, proline, 3-(3-benzothienyl)-alanine, and naphthylalanine, each of which is optionally substituted with one or more substituents. In other embodiments, each hydrophobic amino acid is independently piperidine-2-carboxylate, naphthyl alanine, tryptophan, 3-(3-benzothienyl)-alanine, or phenylalanine, each of which is optionally substituted with one or more substituents. In some embodiments, at least one hydrophobic amino acid has a hydrophobicity greater than or equal to phenylalanine.

In certain embodiments, at least two of the amino acids have the opposite chirality. In further embodiments, at least two of the amino acids have the same chirality.

In some embodiments, one arginine is adjacent to one hydrophobic amino acid. In further embodiments, the arginine has the same chirality as the adjacent hydrophobic amino acid.

In some embodiments, at least two arginines are adjacent to each other. In other embodiments, three arginines are adjacent to each other. In some embodiments, at least two hydrophobic amino acids are adjacent to each other. In other embodiments, at least three hydrophobic amino acids are adjacent to each other.

In some embodiments, any four adjacent amino acids are AA_{H2}-AA_{H1}-R-r, AA_{H2}-AA_{H1}-r-R, R-r-AA_{H1}-AA_{H2}, or r-R-AA_{H1}-AA_{H2}, wherein each of AA_{H1} and AA_{H2} are independently a hydrophobic amino acid.

In some embodiments, the cyclic peptides of Formula III-A to III-D have a structure according to any of Formula IV-A to IV-P:

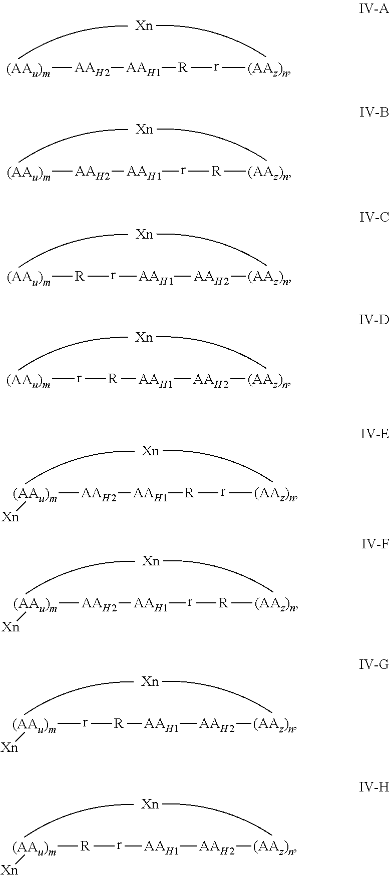

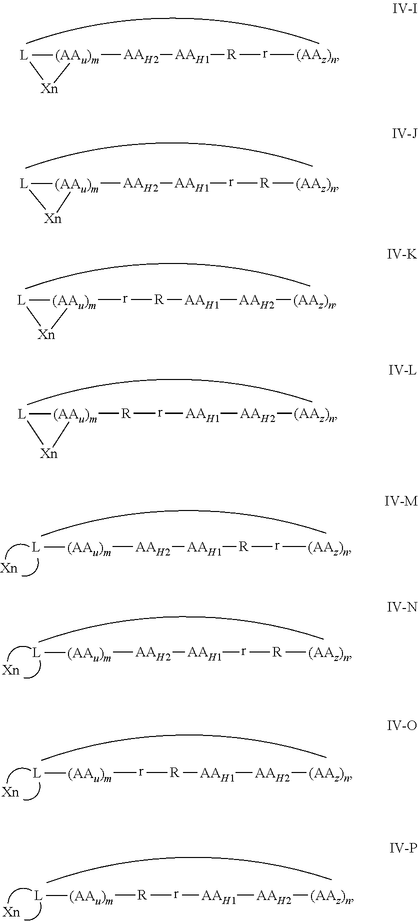

wherein each of AA_{H1} and AA_{H2} are independently a hydrophobic amino acid.

In some embodiments, each of AA_{H1} and AA_{H2} are independently piperidine-2-carboxylate, naphthylalanine, tryptophan, 3-(3-benzothienyl)-alanine, or phenylalanine, each of which is optionally substituted with one or more substituents. In other embodiments, each of AA_{H1} and AA_{H2} are independently a hydrophobic amino acid having a hydrophobicity greater than or equal to phenylalanine (e.g., using any of the scales provided herein in Table 2). In still other embodiments, AA_{H1} is naphthylalanine. In yet still other embodiments, AA_{H1} and AA_{H2} are naphthylalanine.

In some embodiments, AA_{H1} is a hydrophobic amino acid with a side chain having a large solvent-accessible surface area (SASA). In further embodiments, the large SASA is at least about 200 Å². In still further embodiments, the large SASA is in the range of from about 200 Å² to about 1,000 Å². In other embodiments, AA_{H2} is a hydrophobic amino acid with a side chain having a SASA that is less than or equal to the SASA of the side chain of AA_{H1}. In still other embodiments, when any AA_{U} or any AA_{Z} is a hydrophobic amino acid, said hydrophobic amino acid has a side chain with SASA which is less than AA_{H1}.

In some embodiments, AA_{H1} and AA_{H2} have the same or opposite chirality. In some other embodiments, AA_{H1} and AA_{H2} have the opposite chirality. In still other embodiments, AA_{H1} has the same chirality as the adjacent arginine.

In certain embodiments, the peptides disclosed herein comprise one of the following sequences: AA_{H2}-AA_{H1}-R-r-R, AA_{H2}-AA_{H1}-R-r-r, AA_{H2}-AA_{H1}-r-R-R, AA_{H2}-AA_{H1}-r-R-r, R-R-r-AA_{H1}-AA_{H2}, r-R-r-AA_{H1}-AA_{H2}, r-r-R-AA_{H1}-AA_{H2}, or, R-r-R-AA_{H1}-AA_{H2}. In other embodiments, the peptides have one of the following sequences AA_{H2}-LAA_{H1}-R-r-R, AA_{H2}-DAA_{H1}-r-R-r, r-R-r-DAA_{H1}-AA_{H2}, or R-r-R-LAA_{H1}-AA_{H2}.

Also disclosed herein are peptides comprising Formula V-A to V-D:

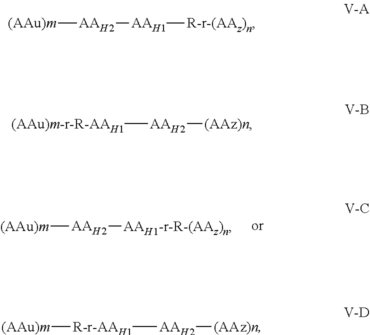

wherein:
each of AA_{H1} and AA_{H2} are independently selected from a hydrophobic amino acid;
at each instance AA_{U} and AA_{Z} are independently selected from an amino acid, with at most one of each AA_{U} and each AA_{Z} being arginine; and
each of m and n are a number from 0 to 6, provided that at least one of m or n is not 0.

In some embodiments, the amino group on the N-terminus or a carboxylate group on the C-terminus of any of Formula V-A to V-D independently form a peptide bond. In other embodiments, the peptides of Formula V-A to V-D further comprise a cyclization moiety which thereby forms a cyclic peptide. In still other embodiments, the cyclization moiety independently forms a peptide bond with an amino group on the N-terminus or a carboxylate group on the C-terminus of any of Formula V-A to V-D. Is some embodiments, the peptides of Formula V-A to V-D further comprise a cargo moiety Xₙ, wherein at least one atom or bond is replaced by a bond to Xₙ.

In some embodiments, each of AA_{H1} and AA_{H2} are independently piperidine-2-carboxylate, naphthylalanine, tryptophan, 3-(3-benzothienyl)-alanine, or phenylalanine, each of which is optionally substituted with one or more substituents. In some embodiments, each of AA_{H1} and AA_{H2} are independently a hydrophobic amino acid having a hydrophobicity greater than or equal to phenylalanine. In some embodiments, AA_{H1} is naphthylalanine. In some embodiments, AA_{H1} and AA_{H2} are naphthylalanine. In some embodiments, AA_{H1} is a hydrophobic amino acid with a side chain having a large solvent-accessible surface area (SASA). In further embodiments, the large SASA is at least about 200 Å². In still further embodiments, the large SASA is in the range of from about 200 Å² to about 1,000 Å². In other embodiments, AA_{H2} is a hydrophobic amino acid with a side chain having a SASA that is less than or equal to the SASA of the side chain of AA_{H1}. In still other embodiments, when any AA_{U} or any AA_{Z} is a hydrophobic amino acid, said hydrophobic amino acid has a side chain with SASA which is less than AA_{H1}.

In some embodiments, AA_{H1} and AA_{H2} have the same or opposite chirality. In some other embodiments, AA_{H1} and AA_{H2} have the opposite chirality. In still other embodiments, AA_{H1} has the same chirality as the adjacent arginine.

In certain embodiments, the peptides of Formula V-A to V-D comprise one of the following sequences: AA_{H2}-AA_{H1}-R-r-R, AA_{H2}-AA_{H1}-R-r-r, AA_{H2}-AA_{H1}-r-R-R, AA_{H2}-AA_{H1}-r-R-r, R-R-r-AA_{H1}-AA_{H2}, r-R-r-AA_{H1}-AA_{H2}, r-r-R-AA_{H1}-AA_{H2}, or, R-r-R-AA_{H1}-AA_{H2}. In other embodiments, the peptides have one of the following sequences AA_{H2}-LAA_{H1}-R-r-R, AA_{H2}-DAA_{H1}-r-R-r, r-R-r-DAA_{H1}-AA_{H2}, or R-r-R-LAA_{H1}-AA_{H2}.

In some embodiments, peptides comprising any of Formula V-A to V-D have a cytosolic delivery efficiency which is improved by an amount in the range of from about 50% to about 500% compared to cyclo(FΦRRRRQ) (SEQ ID NO:10). In some embodiments, such peptides comprise the following sequence: FfFRrR (SEQ ID NO:131). In other embodiments, such peptides comprise the following sequence: fFfrRr (SEQ ID NO:132). In still other embodiments, such peptides comprise the following sequence: fFfRrR (SEQ ID NO:133). In yet still other embodiments, such peptides comprise the following sequence: FfFrRr (SEQ ID NO:134). In even still other embodiments, such peptides comprise the following sequence: fFϕrRr (SEQ ID NO:135). In some embodiments, the peptides comprise any of Formula V-A to V-D comprises the following sequence: fΦfrRr (SEQ ID NO:136).

In various embodiments, the present disclosure provides for methods of treating or diagnosing a patient in need thereof, comprising administering cyclic peptide disclosed herein.

In various embodiments, the present disclosure provides for compositions comprising the cyclic peptide described herein.

### DETAILED DESCRIPTION

### Definitions

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It will also be understood that when a range is provided, said range encompasses each and every value and subrange within the range.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “anticancer” refers to the ability to treat or control cellular proliferation and/or tumor growth at any concentration.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The terms “peptide,” “protein,” and “polypeptide” are used interchangeably to refer to a natural or synthetic molecule comprising two or more amino acids linked by the carboxyl group of one amino acid to the alpha amino group of another.

As used herein, the term “adjacent” refers to two contiguous amino acids which are connected by a covalent bond.

As used herein, the term “chirality” refers to the “D” and “L” isomers of amino acids.

The term “acyl” refers to groups —C(O)R, where R is hydrogen, alkyl, alkenyl, alkynyl, aryl, heteroaryl, cycloalkyl, or heterocyclyl, as defined herein. Unless stated otherwise specifically in the specification, acyl can be optionally substituted.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkoxy” refers to the group —OR, where R is alkyl, alkenyl, alkynyl, cycloalkyl, or heterocyclyl as defined herein. Unless stated otherwise specifically in the specification, alkoxy can be optionally substituted.

“Alkylcarbamoyl” refers to the group —O—C(O)—NRₐR_{b}, where Rₐ and R_{b} are the same or different and independently an alkyl, alkenyl, alkynyl, aryl, heteroaryl, cycloalkyl, or heterocyclyl group, as defined herein, or RₐR_{b} can be taken together to form a heterocyclyl group, as defined herein. Unless stated otherwise specifically in the specification, alkylcarbamoyl can be optionally substituted.

“Alkylcarboxamidyl” refers to the group —C(O)—NRₐR_{b}, where Rₐ and R_{b} are the same or different and independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, alkylcarboxamidyl can be optionally substituted.

“Alkoxycarbonyl” refers to the group —C(O)OR, where R is a alkyl, alkenyl, alkynyl, aryl, heteroaryl, cycloalkyl, cycloalkenyl, cycloalkynyl, or heterocyclyl group, as defined herein. Unless stated otherwise specifically in the specification, alkoxycarbonyl can be optionally substituted.

“Alkylthio” refers to the —SR or —S(O)ₙ₌₁₋₂—R, where R is alkyl, alkenyl, alkynyl, cycloalkyl, cycloalkenyl, cycloalkynyl, or hetereocyclyl, as defined herein. Unless stated otherwise specifically in the specification, alkylthio can be optionally substituted.

“Arylthio” refers to the —SR or —S(O)ₙ₌₁₋₂—R, where R is aryl or hetereoaryl, as defined herein. Unless stated otherwise specifically in the specification, arylthio can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aryloxy” refers to groups —OAr, where Ar is an aryl or heteroaryl group as defined herein. Unless otherwise stated specifically in the specification, the aryloxy group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

### Cell Penetrating Peptides

Disclosed herein are compounds having activity as cell penetrating peptides (CPPs). In some embodiments, the CPPs include any combination of between two or three arginines and at least two hydrophobic amino acids, with a total number of amino acids in the CPP in the range of from 4 to about 20 amino acids. In some embodiments, the CPPs disclosed herein comprise about 4 to about to about 13 amino acids, e.g., about 5, about 6, about 7, about 8, about 9, about 10, or about 11 amino acids, or about 12 amino acids, inclusive of all ranges and subranges therebetween. In particular embodiments, the CPPs disclosed herein comprise about 6 to about 10 amino acids, or about 6 to about 8 amino acids.

Each amino acid can be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Thus, as used herein, the term “amino acid” refers to natural and non-natural amino acids, and analogs and derivatives thereof. Examples of suitable amino acids include, but are not limited to, alanine, allosoleucine, arginine, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative, or combinations thereof. These, and others, are listed in the Table 1 along with their abbreviations used herein.

TABLE 1
Amino Acid Abbreviations

| | Abbrevi- | Abbrevi- |
|---|---|---|
| | ations* | ations* |
| | L-amino | D-amino |
| Amino Acid | acid | acid |
| Alanine | Ala (A) | ala (a) |
| Allosoleucine | AIle | aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Cyclohexylalanine | Cha | cha |
| 2,3-diaminopropionic acid | Dap | dap |
| 4-fluorophenylalanine | Fpa (Σ) | pfa |
| Glutamic acid | Glu (E) | glu (e) |
| Glutamine | Gln (Q) | gln (q) |
| Glycine | Gly (G) | gly (g) |
| Gistidine | His (H) | his (h) |
| Homoproline (aka pipecolic acid) | Pip (Θ) | pip (θ) |
| Isoleucine | Ile (I) | ile (i) |
| Leucine | Leu (L) | leu (1) |
| Lysine | Lys (K) | lys (k) |
| Methionine | Met (M) | met (m) |
| Napthylalanine | Nal (Φ) | nal (ϕ) |
| Norleucine | Nle (Ω) | nle |
| Phenylalanine | Phe (F) | phe(F) |
| Phenylglycine | Phg (Ψ) | phg |
| 4-(phosphonodifluoromethyl)phenylalanine | F₂Pmp (Λ) | f₂pmp |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| 3-(3-benzothienyl)-alanine | Bta | bta |

*single letter abbreviations: when shown in capital letters herein it indicates the L-amino acid form, when shown in lower case herein it indicates the D-amino acid form.

In some embodiments, the present disclosure provides for cyclic peptide having a structure according to Formula I:

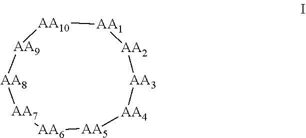

wherein:
each of AA₁, AA₂, AA₃, AA₄, AA₅, and AA₆ are independently selected from an amino acid; and
each of AA₇, AA₈, AA₉, and AA₁₀, are independently absent or selected from an amino acid; and

wherein:
two or three of AA₁, AA₂, AA₃, AA₄, AA₅, AA₆, AA₇, AA₈, AA₉, or AA₁₀ are arginine, with the remaining amino acids thereof being an amino acid other than arginine; and
at least two of AA₁, AA₂, AA₃, AA₄, AA₅, AA₆, AA₇, AA₈, AA₉, or AA₁₀ are independently a hydrophobic amino acid.

The amino acids, and arrangement thereof, of Formula I are described in detail below.

In some embodiments, the CPPs disclosed herein (i.e., the cyclic peptides of Formula I) have a structure according to any of Formula I-A to I-E:

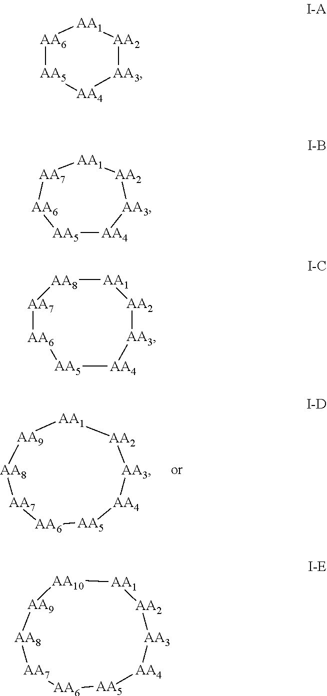

wherein each of AA₁, AA₂, AA₃, AA₄, AA₅, AA₆, AA₇, AA₈, AA₉, and AA₁₀, when present, is independently selected from an amino acid; and
wherein:
   two or three of AA₁, AA₂, AA₃, AA₄, AA₅, AA₆, AA₇, AA₈, AA₉, or AA₁₀, when present, are arginine, with the remaining amino acids thereof being an amino acid other than arginine; and
   at least two of AA₁, AA₂, AA₃, AA₄, AA₅, AA₆, AA₇, AA₈, AA₉, or AA₁₀, when present, are independently a hydrophobic amino acid.

When the cyclic peptide has a structure according to Formula I-A, two or three of AA₁, AA₂, AA₃, AA₄, AA₅, and AA₆ are arginine. When the cyclic peptide has a structure according to Formula I-B, two or three of AA₁, AA₂, AA₃, AA₄, AA₅, AA₆, and AA₇ are arginine. When the cyclic peptide has a structure according to Formula I-C, two or three of AA₁, AA₂, AA₃, AA₄, AA₅, AA₆, AA₇, and AA₈ are arginine. When the cyclic peptide has a structure according to Formula I-D, two or three of AA₁, AA₂, AA₃, AA₄, AA₅, AA₆, AA₇, AA₈, and AA₉ are arginine. When the cyclic peptide has a structure according to Formula I-E, two or three of AA₁, AA₂, AA₃, AA₄, AA₅, AA₆, AA₇, AA₈, AA₉, and AA₁₀ are arginine. Therefore, the cyclic peptide sequences according to Formula I, I-A, I-B, I-C, I-D, and I-E include two or three arginines, but not more than three arginines.

In some embodiments, each hydrophobic amino acid is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylate, 3-(3-benzothienyl)-alanine, or norleucine, each of which is optionally substituted with one or more substituents. In particular embodiments, each hydrophobic amino acid is independently a hydrophobic aromatic amino acid. In some embodiments, the aromatic hydrophobic amino acid is naphthylalanine, phenylglycine, homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The structures of a few of these non-natural aromatic hydrophobic amino acids (prior to incorporation into the peptides disclosed herein) are provided below. In particular embodiments, the hydrophobic amino acid is piperidine-2-carboxylate, naphthylalanine, tryptophan, 3-(3-benzothienyl)-alanine, or phenylalanine, each of which is optionally substituted with one or more substituents.

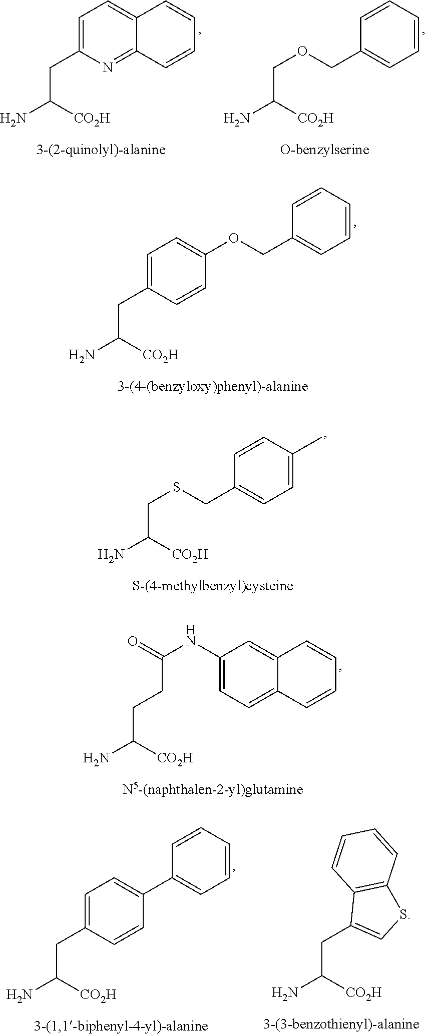

Those skilled in the art will appreciate that the N- and/or C-termini of the above non-natural aromatic hydrophobic amino acids, upon incorporation into the peptides disclosed herein (e.g., the peptides of Formulae I, I-A to I-E, II-A to II-D, III-A to III-D, IV-A to IV-P, and V-A to V-D), form amide bonds.

The optional substituent can be any atom or group which does not significantly reduce the cytosolic delivery efficiency of the CPP, e.g., a substituent that does not reduce the relative cytosolic delivery efficiency to less than that of c(FΦRRRRQ) (SEQ ID NO:10). In some embodiments, the optional substituent can be a hydrophobic substituent or a hydrophilic substituent. In certain embodiments, the optional substituent is a hydrophobic substituent. In some embodiments, the substituent increases the solvent-accessible surface area (as defined herein below) of the hydrophobic amino acid. In some embodiments, the substituent can be a halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. In some embodiments, the substituent is a halogen.

Amino acids having higher hydrophobicity values can be selected to improve cytosolic delivery efficiency of a CPP relative to amino acids having a lower hydrophobicity value. In some embodiments, each hydrophobic amino acid independently has a hydrophobicity value which is greater than that of glycine. In other embodiments, each hydrophobic amino acid independently is a hydrophobic amino acid having a hydrophobicity value which is greater than that of alanine. In still other embodiments, each hydrophobic amino acid independently has a hydrophobicity value which is greater or equal to that of phenylalanine. Hydrophobicity may be measured using hydrophobicity scales known in the art. Table 2 below lists hydrophobicity values for various amino acids as reported by Eisenberg and Weiss (Proc. Natl. Acad. Sci. U.S.A 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A. 1981; 78(6):3824-3828), and Janin (Nature. 1979; 277(5696):491-492), the entirety of each of which is herein incorporated by reference in its entirety. In particular embodiments, hydrophobicity is measured using the hydrophobicity scale reported in Engleman, et al.

TABLE 2

| | | | | | Hoop | |
|---|---|---|---|---|---|---|
| Amino | | Eisenberg | Engleman | Kyrie and | and | |
| Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/ | 0.04 | 2.0 | 2.5 | −1.0 | 0.9 |
| | Polar | | | | | |
| Tyr | Unch/ | 0.02 | −0.7 | −1.3 | −2.3 | −0.4 |
| | Polar | | | | | |
| Pro | Nonpolar | −0.07 | −0.2 | −1.6 | 0.0 | −0.3 |
| Thr | Unch/ | −0.18 | 1.2 | −0.7 | −0.4 | −0.2 |
| | Polar | | | | | |
| Ser | Unch/ | −0.26 | 0.6 | −0.8 | 0.3 | −0.1 |
| | Polar | | | | | |
| His | Charged | −0.40 | −3.0 | −3.2 | −0.5 | −0.1 |
| Glu | Charged | −0.62 | −8.2 | −3.5 | 3.0 | −0.7 |
| Asn | Unch/ | −0.64 | −4.8 | −3.5 | 0.2 | −0.5 |
| | Polar | | | | | |
| Gln | Unch/ | −0.69 | −4.1 | −3.5 | 0.2 | −0.7 |
| | Polar | | | | | |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The chirality of the amino acids can be selected to improve cytosolic uptake efficiency. In some embodiments, at least two of the amino acids have the opposite chirality. In some embodiments, the at least two amino acids having the opposite chirality can be adjacent to each other. In some embodiments, at least three amino acids have alternating chirality relative to each other. In some embodiments, the at least three amino acids having the alternating chirality relative to each other can be adjacent to each other. In some embodiments, at least two of the amino acids have the same chirality. In some embodiments, the at least two amino acids having the same chirality can be adjacent to each other. In some embodiments, at least two adjacent amino acids have the same chirality and at least two adjacent amino acids have the opposite chirality. In some embodiments, the at least two amino acids having the opposite chirality can be adjacent to the at least two amino acids having the same chirality. Accordingly, in some embodiments, adjacent amino acids in the CPP can have any of the following sequences: D-L; L-D; D-L-L-D; L-D-D-L; L-D-L-L-D; D-L-D-D-L; D-L-L-D-L; or L-D-D-L-D.

In some embodiments, an arginine is adjacent to a hydrophobic amino acid. In some embodiments, the arginine has the same chirality as the hydrophobic amino acid. In some embodiments, at least two arginines are adjacent to each other. In other embodiments, three arginines are adjacent to each other. In some embodiments, at least two hydrophobic amino acids are adjacent to each other. In other embodiments, at least three hydrophobic amino acids are adjacent to each other. In other embodiments, the CPPs described herein comprise at least two consecutive hydrophobic amino acids and at least two consecutive arginines. In further embodiments, one hydrophobic amino acid is adjacent to one of the arginines. In still other embodiments, the CPPs described herein comprise at least three consecutive hydrophobic amino acids and three consecutive arginines. In further embodiments, one hydrophobic amino acid is adjacent to one of the arginines. These various combinations of amino acids can have any arrangement of D and L amino acids, e.g., any of the sequences described in the preceding paragraph.

In some embodiments, any four adjacent amino acids in the CPPs described herein (e.g., the CPPs according to Formula IA to I-D) can have one of the following sequences: AA_{H2}-AA_{H1}-R-r, AA_{H2}-AA_{H1}-r-R, R-r-AA_{H1}-AA_{H2}, or r-R-AA_{H1}-AA_{H2}, wherein each of AA_{H1} and AA_{H2} are independently a hydrophobic amino acid.

Accordingly, in some embodiments, the CPPs described herein have a structure according any of Formula II-A to II-D:

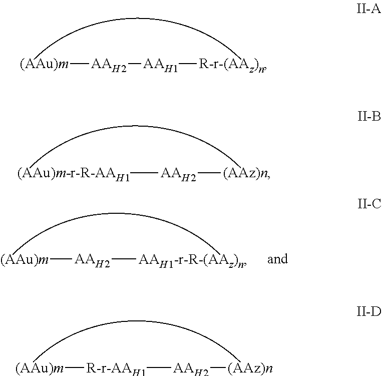

wherein:
each of AA_{H1} and AA_{H2} are independently a hydrophobic amino acid;
at each instance AA_{U} and AA_{Z} are independently any amino acid;
with at most one of each AA_{U} and each AA_{Z} being arginine; and

wherein:
each of m and n are independently a number from 0 to 6, provided that at least one of m or n is not 0 and the total number of amino acids is from 6 to 10.

In some embodiments, the total number of amino acids (including r, R, AA_{H1}, AA_{H2}), in the CPPs of Formula II-A to II-D is in the range of 6 to 10. In some embodiments, the total number of amino acids is 6. In some embodiments, the total number of amino acids is 7. In some embodiments, the total number of amino acids is 8. In some embodiments, the total number of amino acids is 9. In some embodiments, the total number of amino acids is 10.

In some embodiments, the sum of m and n is from 2 to 6. In some embodiments, the sum of m and n is 2. In some embodiments, the sum of m and n is 3. In some embodiments, the sum of m and n is 4. In some embodiments, the sum of m and n is 5. In some embodiments, the sum of m and n is 6. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, each hydrophobic amino acid is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylate, or norleucine, each of which is optionally substituted with one or more substituents. In particular embodiments, each hydrophobic amino acid is independently a hydrophobic aromatic amino acid. In some embodiments, the aromatic hydrophobic amino acid is naphthylalanine, phenylglycine, homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. In particular embodiments, the hydrophobic amino acid is piperidine-2-carboxylate, naphthylalanine, tryptophan, 3-(3-benzothienyl)-alanine, or phenylalanine, each of which is optionally substituted with one or more substituents.

In some embodiments, each of AA_{H1} and AA_{H2} are independently a hydrophobic amino acid having a hydrophobicity value which is greater than that of glycine. In other embodiments, each of AA_{H1} and AA_{H2} are independently a hydrophobic amino acid having a hydrophobicity value which is greater than that of alanine. In still other embodiments, each of AA_{H1} and AA_{H2} are independently an hydrophobic amino acid having a hydrophobicity value which is greater than that of phenylalanine, e.g., as measured using the hydrophobicity scales described above, including Eisenberg and Weiss (Proc. Natl. Acad. Sci. U.S.A 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A. 1981; 78(6):3824-3828), and Janin (Nature. 1979; 277(5696):491-492), (see Table 1 above). In particular embodiments, hydrophobicity is measured using the hydrophobicity scale reported in Engleman, et al.

The presence of a hydrophobic amino acid on the N- or C-terminus of a D-Arg or L-Arg, or a combination thereof, has also been found to improve the cytosolic uptake of the CPP (and the attached cargo). For example, in some embodiments, the CPPs disclosed herein may include AA_{H1}-D-Arg or D-Arg-AA_{H1}. In other embodiments, the CPPs disclosed herein may include AA_{H1}-L-Arg or L-Arg-AA_{H1}. In some embodiments, the presence of the hydrophobic amino acid on the N- or C-terminus of the D-Arg or L-Arg, or a combination thereof, in the CPP improves the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween. In some embodiments, the presence of the hydrophobic amino acid on the N- and/or C-terminus of the D-Arg and/or L-Arg in the CPP improves the cytosolic uptake efficiency by about 20 fold.

The size of the hydrophobic amino acid on the N- or C-terminus of the D-Arg or an L-Arg, or a combination thereof (i.e., AA_{H1}), may be selected to improve cytosolic delivery efficiency of the CPP. For example, a larger hydrophobic amino acid on the N- or C-terminus of a D-Arg or L-Arg, or a combination thereof, improves cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. In some embodiments, the size of the hydrophobic amino acid is measured in terms of the molecular weight of the hydrophobic amino acid, and the larger hydrophobic amino acid has a side chain with a molecular weight of at least about 90 g/mol, or at least about 130 g/mol, or at least about 141 g/mol. In particular embodiments, the size of the amino acid is measured in terms of the SASA of the hydrophobic side chain, and the larger hydrophobic amino acid has a side chain with a SASA greater than that of alanine, or greater than that of glycine. In other embodiments, AA_{H1} has a hydrophobic side chain with a SASA greater than or equal to about piperidine-2-carboxylate, greater than or equal to about tryptophan, greater than or equal to about phenylalanine, or equal to or greater than about naphthylalanine. In some embodiments, AA_{H1} and AA_{H2} independently have a side with a SASA in the range of from about 200 Å² to about 1000 Å², e.g, about 250 Å², 300 Å², 350 Å², 400 Å², 450 Å², 500 Å², 550 Å², 650 Å², 700 Å², 750 Å², 800 Å², 850 Å², 900 Å², and about 950 Å², inclusive of all values and subranges therebetween.

In some embodiments, AA_{H1} has a side chain with a SASA of at least about 200 Å², at least about 210 Å2, at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, AA_{H2} has a side chain side with a SASA of at least about 200 Å², at least about 210 Å2, at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, the side chains of AA_{H1} and AA_{H2} have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². In some embodiments, AA_{H2} is a hydrophobic amino acid with a side chain having a SASA that is less than or equal to the SASA of the hydrophobic side chain of AA_{H1}. By way of example, and not by limitation, a CPP having a Nal-Arg motif exhibits improved cytosolic delivery efficiency compared to an otherwise identical CPP having a Phe-Arg motif; a CPP having a Phe-Nal-Arg motif exhibits improved cytosolic delivery efficiency compared to an otherwise identical CPP having a Nal-Phe-Arg motif; and a phe-Nal-Arg motif exhibits improved cytosolic delivery efficiency compared to an otherwise identical CPP having a nal-Phe-Arg motif. In some embodiments, the presence of the larger hydrophobic amino acid on the N- or C-terminus of the D-Arg or L-Arg, or a combination thereof, in the CPP improves cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween. In particular embodiments, the presence of the larger hydrophobic amino acid on the N- and/or C-terminus of the D-Arg and/or L-Arg in the CPP improves the cytosolic uptake efficiency by about 20 fold.

As used herein, “hydrophobic surface area” or “SASA” refers to the surface area (reported as square Angstroms; Å²) of an amino acid side chain that is accessible to a solvent. In particular embodiments, SASA is calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 3. In certain embodiments, the SASA values described herein are based on the theoretical values listed in Table 3 below, as reported by Tien, et al. (PLOS ONE 8(11): e80635. https://doi.org/10.1371/journal.pone.0080635), which is herein incorporated by reference in its entirety for all purposes.

TABLE 3

| | | | Miller et al. | Rose et al. |
|---|---|---|---|---|
| Residue | Theoretical | Empirical | (1987) | (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

In some embodiments, the CPP does not include a hydrophobic amino acid on the N- and/or C-terminus of AA_{H2}-AA_{H1}-R-r, AA_{H2}-AA_{H1}-r-R, R-r-AA_{H1}-AA_{H2}, or r-R-AA_{H1}-AA_{H2}. In alternative embodiments, the CPP does not include a hydrophobic amino acid having a side chain which is larger (as described herein) than at least one of AA_{H1} or AA_{H2}. In further embodiments, the CPP does not include a hydrophobic amino acid with a side chain having a surface area greater than AA_{H1}. That is, when any AA_{U} or any AA_{Z} is a hydrophobic amino acid (e.g., in Formulae I, I-A to I-E, II-A to II-D, III-A to III-D, IV-A to IV-P, and V-A to V-D), said hydrophobic amino acid has a side chain with SASA which is less than AA_{H1}. For example, in embodiments in which at least one of AA_{H1} or AA_{H2} is phenylalanine, the CPP does not further include a naphthylalanine (although the CPP may include at least one hydrophobic amino acid which is smaller than AA_{H1} and AA_{H2}, e.g., leucine). In still other embodiments, the CPP does not include a naphthylalanine (or a larger hydrophobic amino acid) in addition to the hydrophobic amino acids in AA_{H2}-AA_{H1}-R-r, AA_{H2}-AA_{H1}-r-R, R-r-AA_{H1}-AA_{H2}, or r-R-AA_{H1}-AA_{H2}.

The chirality of the amino acids (i.e., D or L amino acids) can be selected to improve cytosolic delivery efficiency of the CPP (and the attached cargo as described below). In some embodiments, the hydrophobic amino acid on the N- or C-terminus of an arginine (e.g., AA_{H1}, AAᵤ or AA_{z}) has the same or opposite chirality as the adjacent arginine. In some embodiments, AA_{H1} has the opposite chirality as the adjacent arginine. For example, when the arginine is D-arg (i.e. “r”), AA_{H1} is a D-AA_{H1}, and when the arginine is L-Arg (i.e., “R”), AA_{H1} is a L-AA_{H1}. Accordingly, in some embodiments, the CPPs disclosed herein may include at least one of the following motifs: D-AA_{H1}-D-arg, D-arg-D-AA_{H1}, L-AA_{H1}-L-Arg, or L-Arg-LAA_{H1}. In particular embodiments, when arginine is D-arg, AA_{H1} can be D-pip, D-nal, D-trp, D-bta, or D-phe. In another non-limiting example, when arginine is L-Arg, AA_{H1} can be L-Pip, L-Nal, L-Trp, L-Bta, or L-Phe. In some embodiments, the presence of the hydrophobic amino acid having the same chirality as the adjacent arginine improves cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold inclusive of all values and subranges therebetween. In some embodiments, the presence of the hydrophobic amino acid having the same chirality as the adjacent arginine improves the cytosolic uptake efficiency by about 2.5 fold.

In some embodiments, the CPPs described herein include three arginines. Accordingly, in some embodiments, the CPPs described herein include one of the following sequences: AA_{H2}-AA_{H1}-R-r-R, AA_{H2}-AA_{H1}-R-r-r, AA_{H2}-AA_{H1}-r-R-R, AA_{H2}-AA_{H1}-r-R-r, R-R-r-AA_{H1}-AA_{H2}, r-R-r-AA_{H1}-AA_{H2}, r-r-R-AA_{H1}-AA_{H2}, or, R-r-R-AA_{H1}-AA_{H2}. In particular embodiments, the CPPS have one of the following sequences AA_{H2}-AA_{H1}-R-r-R, AA_{H2}-AA_{H1}-r-R-r, r-R-r-AA_{H1}-AA_{H2}, or R-r-R-AA_{H1}-AA_{H2}. In some embodiments, the chirality of AAH₁ and AAH₂ can be selected to improve cytosolic uptake efficiency, e.g., as described above, where AAH₁ has the same chirality as the adjacent arginine, and AAH₁ and AAH₂ have the opposite chirality.

In some embodiments, the CPPs described herein include three hydrophobic amino acids. Accordingly, in some embodiments, the CPPs described herein include one of the following sequences: AA_{H3}-AA_{H2}-AA_{H1}-R-r, AA_{H3}-AA_{H2}-AA_{H1}-R-r, AA_{H3}-AA_{H2}-AA_{H1}-r-R, AA_{H3}-AA_{H2}-AA_{H1}-r-R, R-r-AA_{H1}-AA_{H2}-AA_{H3}, R-r-AA_{H1}-AA_{H2}-AA_{H3}, r-R-AA_{H1}-AA_{H2}-AA_{H3}, or, r-R-AA_{H1}-AA_{H2}-AA_{H3}, wherein AA_{H3} is any hydrophobic amino acid described above, e.g., piperidine-2-carboxylate, naphthyl alanine, tryptophan, 3-(3-benzothienyl)-alanine, or phenylalanine. In some embodiments, the chirality of AA_{H1}, AA_{H2}, and AA_{H3} can be selected to improve cytosolic uptake efficiency, e.g., as described above, where AAH₁ has the same chirality as the adjacent arginine, and AA_{H1} and AA_{H2} have the opposite chirality. In other embodiments, the size of AA_{H1}, AA_{H2}, and AA_{H3} can be selected to improve cytosolic uptake efficiency, e.g., as described above, where AA_{H3} has a SAS of less than or equal to AA_{H1} and separately/or AA_{H2}.

In some embodiments, AA_{H1} and AA_{H2} have the same or opposite chirality. In certain embodiments, AA_{H1} and AA_{H2} have the opposite chirality. Accordingly, in some embodiments, the CPPs disclosed herein include at least one of the following sequences: D-AA_{H2}-L-AA_{H1}-R-r; L-AA_{H2}-D-AA_{H1}-r-R; R-r-D-AA_{H1}-L-AA_{H2}; or r-R-L-AA_{H1}-D-AA_{H1}, wherein each of D-AA_{H1} and D-AA_{H2} is a hydrophobic amino acid having a D configuration, and each of L-AA_{H1} and L-AA_{H2} is a hydrophobic amino acid having an L configuration. In some embodiments, each of D-AA_{H1} and D-AA_{H2} is independently selected from the group consisting of D-pip, D-nal, D-trp, D-bta, and D-phe. In particular embodiments, D-AA_{H1} or D-AA_{H2} is D-nal. In other particular embodiments, D-AA_{H1} is D-nal. In some embodiments, each of L-AA_{H1} and L-AA_{H2} is independently selected from the group consisting of L-Pip, L-Nal, L-Trp, L-Bta, and L-Phe. In particular embodiments, each of L-AA_{H1} and L-AA_{H2} is L-Nal. In other particular embodiments, L-AA_{H1} is L-Nal. In some embodiments, the presence of an AA_{H1} and AA_{H2} having an opposite chirality improves the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween. In some embodiments, the presence of an AA_{H1} and AA_{H2} having an opposite chirality improves cytosolic delivery efficiency by about 1.5 fold.

As discussed above, the disclosure provides for various modifications to a cyclic peptide sequence which may improve cytosolic delivery efficiency. In some embodiments, improved cytosolic uptake efficiency can be measured by comparing the cytosolic delivery efficiency of the CPP having the modified sequence to a proper control sequence. In some embodiments, the control sequence does not include a particular modification (e.g., matching chirality of R and AA_{H1}) but is otherwise identical to the modified sequence. In other embodiments, the control has the following sequence: cyclic (FΦRRRRQ) (SEQ ID NO:10).

As used herein cytosolic delivery efficiency refers to the ability of a CPP to traverse a cell membrane and enter the cytosol. In embodiments, cytosolic delivery efficiency of the CPP is not dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a CPP (or a CPP-cargo conjugate) over the concentration of the CPP (or the CPP-cargo conjugate) in the growth medium. Relative cytosolic delivery efficiency refers to the concentration of a CPP in the cytosol compared to the concentration of a control CPP in the cytosol. Quantification can be achieved by fluorescently labeling the CPP (e.g., with a FTIC dye) and measuring the fluorescence intensity using techniques well-known in the art.

In particular embodiments, relative cytosolic delivery efficiency is determined by comparing (i) the amount of a CPP of the invention internalized by a cell type (e.g., HeLa cells) to (ii) the amount of the control CPP internalized by the same cell type. To measure relative cytosolic delivery efficiency, the cell type may be incubated in the presence of a cell-penetrating peptide of the invention for a specified period of time (e.g., 30 minutes, 1 hour, 2 hours, etc.) after which the amount of the CPP internalized by the cell is quantified using methods known in the art, e.g., fluorescence microscopy. Separately, the same concentration of the control CPP is incubated in the presence of the cell type over the same period of time, and the amount of the control CPP internalized by the cell is quantified.

In other embodiments, relative cytosolic delivery efficiency can be determined by measuring the IC₅₀ of a CPP having a modified sequence for an intracellular target, and comparing the IC₅₀ of the CPP having the modified sequence to a proper control sequence (as described herein).

In some embodiments, the relative cytosolic delivery efficiency of the CPPs described herein is in the range of from about 50% to about 450% compared to cyclo(FΦRRRRQ) (SEQ ID NO:10), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween. In other embodiments, the relative cytosolic delivery efficiency of the CPPs described herein is improved by greater than about 600% compared to cyclo(FΦRRRRQ) (SEQ ID NO:10). In certain embodiments, the CPP comprises FfFRrR (SEQ ID NO:131), e.g., cyclo(FfFRrRQ) (SEQ ID NO:96), and has a relative cytosolic delivery efficiency of 175% to about 250% compared to cyclo(FΦRRRRQ) (SEQ ID NO:10). In certain embodiments, the CPP comprises fFϕrRr (SEQ ID NO:132), e.g., cyclo(fFϕrRrQ) (SEQ ID NO:97), and has a relative cytosolic delivery efficiency of about 150% to about 400% compared to cyclo(FΦRRRRQ) (SEQ ID NO:10). In certain embodiments, the CPP comprises fFfRrR (SEQ ID NO:133) e.g., cyclo(fFfRrRQ) (SEQ ID NO:98), and has a relative cytosolic delivery efficiency of about 75% to about 275% compared to cyclo(FΦRRRRQ) (SEQ ID NO:10). In certain embodiments, the CPP comprises FfFrRr (SEQ ID NO:134), e.g., cyclo(FfFrRrQ) (SEQ ID NO:99), and has a relative cytosolic delivery efficiency of about 150% to about 250% compared to cyclo(FΦRRRRQ) (SEQ ID NO:10). In certain embodiments, the CPP comprises fFϕrRr (SEQ ID NO:135), e.g., cyclo(fFϕrRrQ) (SEQ ID NO:100), and has a relative cytosolic delivery efficiency which is improved by about 200% to about 450% compared to cyclo(FΦRRRRQ) (SEQ ID NO:10). In certain embodiments, the CPP comprises fΦfrRr (SEQ ID NO:136), e.g., cyclo(fΦfrRrQ) (SEQ ID NO:101), and has a relative cytosolic delivery efficiency which is improved by about 250% to about 450% compared to cyclo(FΦRRRRQ) (SEQ ID NO:10).

In other embodiments, the absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween. In certain embodiments, the CPP is cyclo(FfFRrRQ) (SEQ ID NO:96), and has an absolute cytosolic delivery efficiency of 40% to about 50%. In certain embodiments, the CPP is cyclo(fFϕrRrQ) (SEQ ID NO:97), and has an absolute cytosolic delivery efficiency of about 50% to about 70%. In certain embodiments, the CPP is cyclo(fFfRrRQ) (SEQ ID NO:98), and has an absolute cytosolic delivery efficiency of about 30% to about 60%. In certain embodiments, the CPP is cyclo(FfFrRrQ) (SEQ ID NO:99), and has an absolute cytosolic delivery efficiency of about 40% to about 55%. In certain embodiments, the CPP is cyclo(fFϕrRrQ) (SEQ ID NO:100), and has an absolute cytosolic delivery efficiency of about 55% to about 75%. In certain embodiments, the CPP is cyclo(fΦfrRrQ) (SEQ ID NO:101), and has an absolute cytosolic delivery efficiency of about 60% to about 80%.

In some embodiments, the CPPs disclosed herein (e.g., Formulae I, I-A to I-E, II-A to II-D, III-A to III-D, IV-A to IV-P, and V-A to V-D) are selected from the sequences provided in Table 4 below.

TABLE 4

| | | | Relative Cytosolic |
|---|---|---|---|
| CPP ID NO | SEQ ID NO | Peptide Sequence^{a} | Delivery Efficiency |
| SAR 1 | SEQ ID NO:10 | cyclo(FΦRRRQ) | 100 ± 6 |
| SAR 19 | SEQ ID NO:58 | cyclo(FFRRRQ) | 89 ± 34 |
| SAR 20 | SEQ ID NO:59 | cyclo(FFrRrQ) | 118 ± 5 |
| SAR 21 | SEQ ID NO:60 | cyclo(FFRrRQ) | 59 ± 7 |
| SAR 22 | SEQ ID NO:61 | cyclo(FRFRRQ) | 15 ± 3 |
| SAR 23 | SEQ ID NO:62 | cyclo(FRRFRQ) | 38 ± 10 |
| SAR 24 | SEQ ID NO:63 | cyclo(FRRRFQ) | 25 ± 4 |
| SAR 25 | SEQ ID NO:64 | cyclo(GΦRRRQ) | 49 ± 5 |
| SAR 26 | SEQ ID NO:65 | cyclo(FFFRAQ) | 52 ± 2 |
| SAR 27 | SEQ ID NO:66 | cyclo(FFFRRQ) | 125 ± 25 |
| SAR 28 | SEQ ID NO:67 | cyclo(FFRRRRQ) | 89 ± 27 |
| SAR 29 | SEQ ID NO:68 | cyclo(FRRFRRQ) | 31 ± 3 |
| SAR 30 | SEQ ID NO:69 | cyclo(FRRRFRQ) | 20 ± 3 |
| SAR 31 | SEQ ID NO:70 | cyclo(RFFRRRQ) | 63 ± 19 |
| SAR 32 | SEQ ID NO:71 | cyclo(RFRRFRQ) | 94 ± 9 |
| SAR 33 | SEQ ID NO:72 | cyclo(FRFRRRQ) | 132 ± 57 |
| SAR 34 | SEQ ID NO:73 | cyclo(FFFRRRQ) | 231 ± 43 |
| SAR 35 | SEQ ID NO:74 | cyclo(FFRRRFQ) | 158 ± 21 |
| SAR 36 | SEQ ID NO:75 | cyclo(FRFFRRQ) | 142 ± 25 |
| SAR 37 | SEQ ID NO:76 | cyclo(RRFFFRQ) | 172 ± 24 |
| SAR 38 | SEQ ID NO:77 | cyclo(FFRFRRQ) | 106 ± 22 |
| SAR 39 | SEQ ID NO:78 | cyclo(FFRRFRQ) | 86 ± 12 |
| SAR 40 | SEQ ID NO:79 | cyclo(FRRFFRQ) | 109 ± 11 |
| SAR 41 | SEQ ID NO:80 | cyclo(FRRFRFQ) | 101 ± 16 |
| SAR 42 | SEQ ID NO:81 | cyclo(FRFRFRQ) | 105 ± 14 |
| SAR 43 | SEQ ID NO:82 | cyclo(RFFRFRQ) | 96 ± 31 |
| SAR 44 | SEQ ID NO:83 | cyclo(GΦRRRQ) | 55 ± 5 |
| SAR 45 | SEQ ID NO:84 | cyclo(FFFRRRRQ) | 122 ± 20 |
| SAR 46 | SEQ ID NO:85 | cyclo(RFFRRRRQ) | 104 ± 5 |
| SAR 47 | SEQ ID NO:86 | cyclo(RRFFRRRQ) | 115 ± 20 |
| SAR 48 | SEQ ID NO:87 | cyclo(RFFFRRRQ) | 167 ± 10 |
| SAR 49 | SEQ ID NO:88 | cyclo(RRFFFRRQ) | 112 ± 20 |
| SAR 50 | SEQ ID NO:89 | cyclo(FFRRFRRQ) | 46 ± 2 |
| SAR 51 | SEQ ID NO:90 | cyclo(FFRRRRFQ) | 162 ± 16 |
| SAR 52 | SEQ ID NO:91 | cyclo(FRRFFRRQ) | 127 ± 22 |
| SAR 53 | SEQ ID NO:92 | cyclo(FFFRRRRRQ | 145 ± 27 |
| SAR 54 | SEQ ID NO:93 | cyclo(FFFRRRRRR | 141 ± 10 |
| SAR 55 | SEQ ID NO:94 | cyclo(FΦRrRrQ) | 212 ± 42 |
| SAR 56 | SEQ ID NO:95 | cyclo(XXRRRRQ) | 165 ± 22 |
| SAR 57 | SEQ ID NO:96 | cyclo(FfFRrRQ) | 218 ± 9 |
| SAR 58 | SEQ ID NO:97 | cyclo(fFfrRrQ) | 280 ± 106 |
| SAR 59 | SEQ ID NO:98 | cyclo(fFfRrRQ) | 185 ± 91 |
| SAR 60 | SEQ ID NO:99 | cyclo(FfFrRrQ) | 205 ± 30 |
| SAR 61 | SEQ ID NO:100 | cyclo(fFΦrRrQ) | 330 ± 101 |
| SAR 62 | SEQ ID NO:101 | cyclo(fΦfrRrQ) | 357 ± 59 |
| SAR 63 | SEQ ID NO:102 | cyclo(ΦFfrRrQ) | 50 ± 9 |
| SAR 64 | SEQ ID NO:103 | cyclo(FΦrRrQ) | 63 ± 11 |
| SAR 65 | SEQ ID NO:104 | cyclo(fΦrRrQ) | 172 ± 37 |
| SAR 66 | SEQ ID NO:105 | Ac-(Lys-fFRrRrD) | 129 ± 9 |
| SAR 67 | SEQ ID NO:106 | Ac-(Dap-fFRrRrD) | 126 ± 33 |
| SAR 68 | SEQ ID NO:107 | 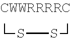 | 51 |
| SAR 69 | SEQ ID NO:108 | 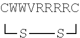 | 67 |
| SAR 70 | SEQ ID NO:109 | 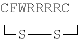 | 49 |
| SAR 71 | SEQ ID NO:110 | 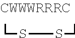 | 135 |
| Pin1 15 | SEQ ID NO:111 | cyclo(Pip-Nal-Arg- | 7.7 ± 0.5 |
| | | Glu-arg-arg-glu) | |
| Pin1 16 | SEQ ID NO:112 | cyclo(Pip-Nal-Arg- | 18 ± 2 |
| | | Arg-arg-arg-glu) | |
| Pin1 17 | SEQ ID NO:113 | cyclo(Pip-Nal-Nal- | 506 ± 120 |
| | | Arg-arg-arg-glu) | |
| Pin1 18 | SEQ ID NO:114 | cyclo(Pip-Nal-Nal- | 340 ± 130 |
| | | Arg-arg-arg-Glu) | |
| Pin1 19 | SEQ ID NO:115 | cyclo(Pip-Nal-Phe- | 19 ± 16 |
| | | Arg-arg-arg-glu) | |
| Pin1 20 | SEQ ID NO:116 | cyclo(Pip-Nal-Phe- | 15 ± 6 |
| | | Arg-arg-arg-Glu) | |
| Pin1 21 | SEQ ID NO:117 | cyclo(Pip-Nal-phe- | 27 ± 15 |
| | | Arg-arg-arg-glu) | |
| Pin1 22 | SEQ ID NO:118 | cyclo(Pip-Nal-phe- | 15 ± 6 |
| | | Arg-arg-arg-Glu) | |
| Pin1 23 | SEQ ID NO:119 | cyclo(Pip-Nal-nal- | 215 ± 86 |
| | | Arg-arg-arg-Glu) | |
| Pin1 24 | SEQ ID NO:120 | cyclo(Pip-Nal-nal- | 141 ± 64 |
| | | Arg-arg-arg-glu) | |

^{a}Φ, L-2-naphthylalanine; Φ, D-2-naphthylalanine; f, D-phenylalanine; r, D-arginine; X, L-4-fluorophenylalanine; Dap, L-2,3-diaminopropionic acid.
^{b}All values are relative to that of CPP 1 (100%) and represent the mean ± S.D. of three independent experiments.

In some embodiments, the CPP sequences disclosed herein (i.e., the sequences according to Formulae I, I-A to I-E, II-A to II-D, III-A to III-D, IV-A to IV-P, and V-A to V-D) do not include sequences disclosed in PCT/US2015/032043, e.g., SEQ ID NO:10 c(FΦRRRRQ); SEQ ID NO:120 c(FfΦRrRrQ); SEQ ID NO:94c(fΦRrRrQ); SEQ ID NO:121 c(fΦRrRrRQ); SEQ ID NO:122c(FϕrRrRq); SEQ ID NO:123c(FϕrRrRQ); SEQ ID NO:124c(FΦRRRRRQ); SEQ ID NO:125 c(RRFRΦRQ); SEQ ID NO:126 c(FFΦRRRRQ); SEQ ID NO:127 c(RFRFRΦRQ); SEQ ID NO:128 c(FΦRRRQ); SEQ ID NO:129 c(FRRRRΦQ); SEQ ID NO:130 c(rRFRΦRQ); or SEQ ID NO:131 c(RRΦFRRQ).

### Cargo

In some embodiments, the CPPs disclosed herein can further include a cargo moiety, which may comprise a peptide (“Xₙ”). The cargo moiety can comprise one or more detectable moieties, one or more therapeutic moieties, one or more targeting moieties, or any combination thereof. In some embodiments, the cargo moiety may be a peptide sequence or a non-peptidyl therapeutic agent. In some embodiments, the cargo moiety can be coupled to an amino group (e.g., N-terminus), a carboxylate group (e.g., C-terminus), or a side chain of one or more amino acids in the CPP. In some embodiments, the CPP and the cargo moiety together are cyclic (referred to herein as “endocyclic”). In some embodiments, the CPP is cyclic and the cargo moiety is appended to the cyclic cell penetrating peptide moiety structure (referred to herein as “exocyclic”). In some embodiments, the cargo moiety is cyclic and the CPP is cyclic, and together they form a bicyclic system (referred to herein as “bicyclic”).

In certain embodiments, at least one bond in the CPP (e.g., an amide bond between amino acid) is replaced by a bond to Xₙ to form an endocyclic CPP and cargo moiety. In certain embodiments, Xₙ is coupled to a side chain of an amino acid in the CPP (e.g., cysteine, lysine, glutamine, or asparagine, or analogs thereof), forming an exocyclic CPP and Xₙ. In certain embodiments, the CPP further comprises a linker group (“L”), and Xₙ is attached to the linker group, forming a bicyclic CPP and cargo moiety. In certain embodiments, the CPP further comprises a linker group (“L”), and Xₙ is attached to the linker group and a side chain of an amino acid of the CPP, forming a bicyclic CPP and cargo moiety.

It is also disclosed herein that for the endocyclic structure, some amino acids in the CPP can also be part of the cargo moiety. For example, a peptide penetrating moiety FNalRR can be formed from FNal and a cargo moiety comprising two Args. In this case, the two Arg residues perform dual functions. Thus, in some cases the sequence of the cargo moiety is taken into account when referring to the peptide penetrating moiety.

In some embodiments, the CPPs disclosed herein have a structure according to one of Formula III-A to III-D:

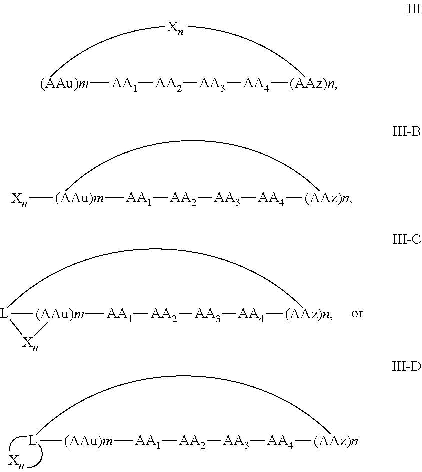

wherein:
each of AA₁, AA₂, AA₃, and AA₄, are independently selected from an amino acid;
at each instance AA_{U} and AA_{Z} are independently selected from an amino acid;
each of m and n are a number from 0 to 6, provided that at least one of m or n is not 0;
Xₙ is a cargo moiety comprising a therapeutic moiety, a targeting moiety, a detectable moiety, or combinations thereof;
L is a linker moiety;

wherein:
two or three of AA₁, AA₂, AA₃, AA₄, each AA_{U}, and each AA_{Z} are arginine, with the remaining amino acids thereof being an amino acid other than arginine;
at least two of AA₁, AA₂, AA₃, AA₄, each AA_{U}, and each AA_{Z} at each instance are independently a hydrophobic amino acid; and

wherein when Xₙ is attached to AA_{U}, m is not 0;

In some embodiments, each of AA₁, AA₂, AA₃, and AA₄, are independently selected from a natural or non-natural amino acid, such as, but not limited to, those described above in Table 1. In some embodiments, each of AA_{U} and AA_{Z} are independently selected from an amino acid, such as, but not limited to, those described above in Table 1.

In some embodiments, at least two of AA₁, AA₂, AA₃, AA₄, each AA_{U}, and each AA_{Z} are independently a hydrophobic amino acid. In some embodiments, each hydrophobic amino acid is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylate, 3-(3-benzothienyl)-alanine, or norleucine, each of which is optionally substituted with one or more substituents. In particular embodiments, each hydrophobic amino acid is independently a hydrophobic aromatic amino acid. In some embodiments, the aromatic hydrophobic amino acid is naphthylalanine, phenylglycine, homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. In particular embodiments, the hydrophobic amino acid is piperidine-2-carboxylate, naphthylalanine, tryptophan, 3-(3-benzothienyl)-alanine, or phenylalanine, each of which is optionally substituted with one or more substituents.

As described above, the optional substituent can be any atom or group which does not significantly reduce the cytosolic delivery efficiency of the CPP, e.g., a substituent that does not reduce relative cytosolic delivery efficiency to less than that of SEQ ID NO:10 c(FΦRRRRQ). In some embodiments, the optional substituent can be a hydrophobic substituent or a hydrophilic substituent. In certain embodiments, the optional substituent is a hydrophobic substituent. In some embodiments, the substituent increases the solvent-accessible surface area (as defined herein) of the hydrophobic amino acid. In some embodiments, the substituent can be a halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. In some embodiments, the substituent is a halogen.

As discussed above, amino acids having higher hydrophobicity values can be selected to improve cytosolic delivery efficiency of a CPP relative to amino acids having a lower hydrophobicity value. In some embodiments, each hydrophobic amino acid independently has a hydrophobicity value which is greater than that of glycine. In other embodiments, each hydrophobic amino acid independently has a hydrophobicity value which is greater than that of alanine (see Table 2, above). In still other embodiments, each hydrophobic amino acid independently has a hydrophobicity value which is greater or equal to phenylalanine (see Table 2, above).

As discussed above, the chirality of the amino acids can be selected to improve cytosolic uptake efficiency. In some embodiments, at least two of the amino acids have the opposite chirality. In some embodiments, the at least two amino acids having the opposite chirality can be adjacent to each other. In some embodiments, at least three amino acids have alternating stereochemistry relative to each other. In some embodiments, the at least three amino acids having the alternating chirality relative to each other can be adjacent to each other. In some embodiments, at least two of the amino acids have the same chirality. In some embodiments, the at least two amino acids having the same chirality can be adjacent to each other. In some embodiments, at least two amino acids have the same chirality and at least two amino acids have the opposite chirality. In some embodiments, the at least two amino acids having the opposite chirality can be adjacent to the at least two amino acids having the same chirality. Accordingly, in some embodiments, adjacent amino acids in the CPP can have any of the following sequences: D-L; L-D; D-L-L-D; L-D-D-L; L-D-L-L-D; D-L-D-D-L; D-L-L-D-L; or L-D-D-L-D.

In some embodiments, an arginine is adjacent to a hydrophobic amino acid. In some embodiments, the arginine has the same chirality as the hydrophobic amino acid. In some embodiments, at least two arginines are adjacent to each other. In still other embodiments, three arginines are adjacent to each other. In some embodiments, at least two hydrophobic amino acids are adjacent to each other. In other embodiments, at least three hydrophobic amino acids are adjacent to each other. In other embodiments, the CPPs described herein comprise at least two consecutive hydrophobic amino acids and at least two consecutive arginines. In further embodiments, one hydrophobic amino acid is adjacent to one of the arginines. In still other embodiments, the CPPs described herein comprise at least three consecutive hydrophobic amino acids and three consecutive arginines. In further embodiments, one hydrophobic amino acid is adjacent to one of the arginines. These various combinations of amino acids can have any arrangement of D and L amino acids, e.g., the sequences described above.

In some embodiments, the sum of m and n is from 2 to 6. In some embodiments, the sum of m and n is 2. In some embodiments, the sum of m and n is 3. In some embodiments, the sum of m and n is 4. In some embodiments, the sum of m and n is 5. In some embodiments, the sum of m and n is 6. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, the linker (“L”) can be any moiety that is capable of forming two peptide bonds and which also contains at least one functional group suitable for attachment of the cargo moiety (Xₙ). In some embodiments, the linker can be one or more natural or non-natural amino acids. Non-limiting examples of a natural amino acid which can function as a linker is glutamine, lysine, and cysteine, and analogs thereof. In certain embodiments, the linker has one functional group for attachment of the cargo moiety, and the cargo moiety also binds to a side chain of an amino acid in the CPP (e.g., glutamine, asparagine, 2,3-diaminopropionic acid, or lysine, or analogs thereof) to form the second cycle of the bicyclic system. In some embodiment, the linker contains at least two functional groups for attachment of the cargo moiety.

In some embodiments, the linker can have the following structure prior to incorporation into the CPP

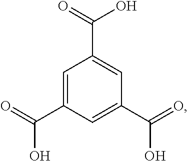 [SMILES: O=C(O)c1cc(C(=O)O)cc(C(=O)O)c1]

wherein each bond to an —OH group is independently replaced by a peptide bond to an amino group of the CPP or cargo.

In other embodiments, the linker can have one of the following structure prior to incorporation into the CPP:

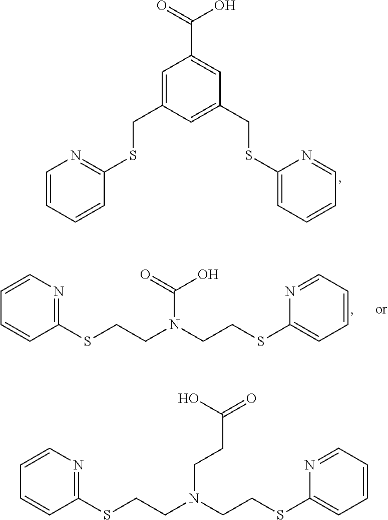 [SMILES: O=C(O)c1cc(CSc2ccccn2)cc(CSc2ccccn2)c1 ; O=C(O)N(CCSc1ccccn1)CCSc1ccccn1 ; O=C(O)CCN(CCSc1ccccn1)CCSc1ccccn1]

wherein each bond to an —OH group is independently replaced by a peptide bond to an amino group of the CPP, and each thioether bond between the aryl group and the sulfur is independently replaced by a disulfide bond with the cargo and, and optionally with the CPP, provided that each of the cargo and the CPP independently include a cysteine or other thiol-containing non-natural amino acid, e.g., those disclosed in U.S. Provisional Application No. 62/438,141.

In some embodiments, Xn is a cargo moiety (as described above) which comprises a therapeutic moiety, a targeting moiety, a detectable moiety, or combinations thereof. Non-limiting examples of therapeutic moieties, targeting moieties, and detectable moieties which can be attached to the CPPs described herein are described below.

In some embodiments, cyclic peptides described herein (e.g., according to Formula III-A to III-D) have a structure according to Formula IV-A to IV-P:

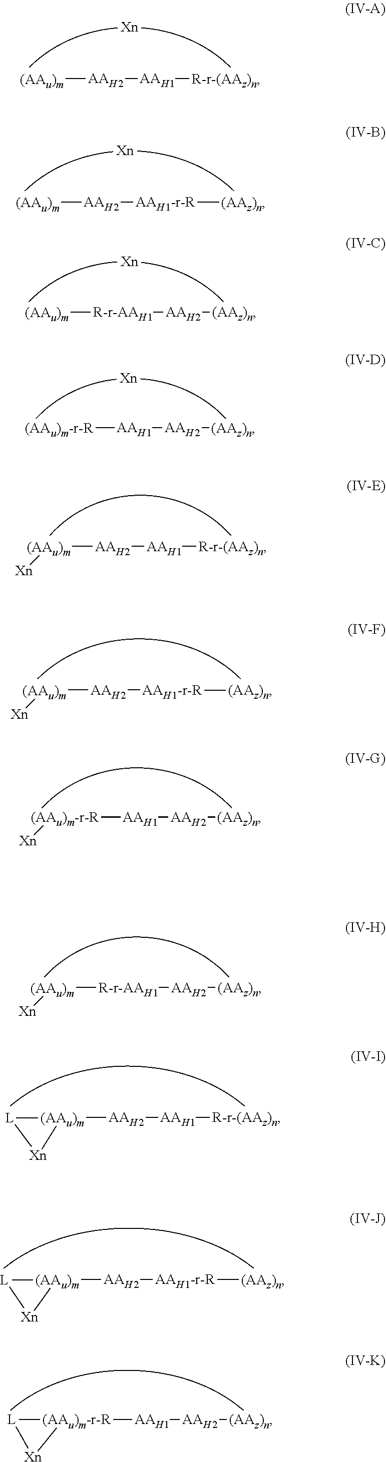

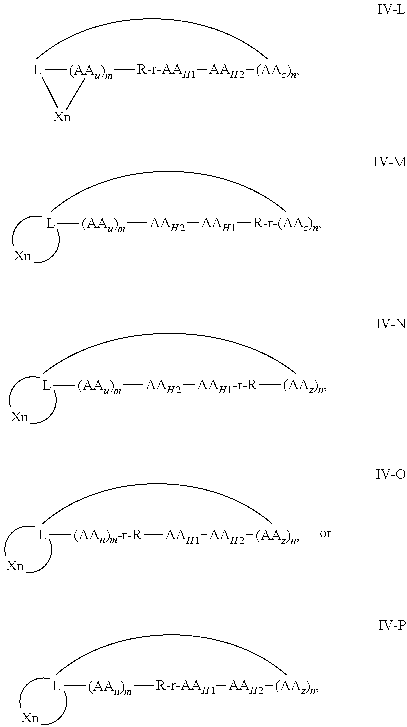

wherein:

each AA_{U} and AA_{Z} are independently any amino acid, e.g., any of AA_{U} at each instance or AA_{Z} at each instance as defined above for Formula III-A to III-D;

each of AA_{H1} and AA_{H2} are independently a hydrophobic amino acid; and

with at most of each AA_{U} and each AA_{Z} being arginine;

each of m and n are independently a number from 0 to 6, provided that at least one of m or n is not 0 and the total number of amino acids is from 6 to 10.

In some embodiments, the total number of amino acids (including r, R, AA_{H1}, AA_{H2}), in the CPPs of Formula IV-A to IV-P are in the range of 6 to 10. In some embodiments, the total number of amino acids is 6. In some embodiments, the total number of amino acids is 7. In some embodiments, the total number of amino acids is 8. In some embodiments, the total number of amino acids is 9. In some embodiments, the total number of amino acids is 10.

In some embodiments, the sum of m and n is from 2 to 6. In some embodiments, the sum of m and n is 2. In some embodiments, the sum of m and n is 3. In some embodiments, the sum of m and n is 4. In some embodiments, the sum of m and n is 5. In some embodiments, the sum of m and n is 6. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, each of AA_{H1} and AA_{H2} are independently a hydrophobic amino acid having a hydrophobicity value which is greater than that of glycine. In other embodiments, each of AA_{H1} and AA_{H2} are independently a hydrophobic amino acid having a hydrophobicity value which is greater than that of alanine. In still other embodiments, each of AA_{H1} and AA_{H2} are independently an hydrophobic amino acid having a hydrophobicity value which is greater than that of phenylalanine, e.g., as measured using the hydrophobicity scales described above, including Eisenberg and Weiss (Proc. Natl. Acad. Sci. U.S.A 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A. 1981; 78(6):3824-3828), and Janin (Nature. 1979; 277(5696):491-492), (see Table 1 above). In particular embodiments, hydrophobicity is measured using the hydrophobicity scale reported in Engleman, et al.

As described above, the presence of a hydrophobic amino acid on the N- or C-terminus of a D-Arg or L-Arg, or a combination thereof, has also found to improve the cytosolic uptake of the CPP (and the attached cargo). For example, in some embodiments, the CPPs disclosed herein may include AA_{H1}-D-Arg or D-Arg-AA_{H1}. In other embodiments, the CPPs disclosed herein may include AA_{H1}-L-Arg or L-Arg-AA_{H1}. In some embodiments, the presence of the hydrophobic amino acid on the N- or C-terminus of the D-Arg or L-Arg, or a combination thereof, in the CPP improves the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

As discussed above, the size of the hydrophobic amino acid on the N- or C-terminus of the D-Arg or an L-Arg, or a combination thereof (i.e., AA_{H1}), may be selected to improve cytosolic delivery efficiency of the CPP. For example, a larger hydrophobic amino acid on the N- or C-terminus of a D-Arg or L-Arg, or a combination thereof, improves cytosolic delivery efficiency compared to an otherwise identical sequence having a smaller hydrophobic amino acid. As discussed above, the size of the hydrophobic amino acid can be measured in terms of molecular weight of the hydrophobic amino acid, the steric effects of the hydrophobic amino acid, the solvent-accessible surface area (SASA) of the side chain, or combinations thereof. In some embodiments, the size of the hydrophobic amino acid is measured in terms of the molecular weight of the hydrophobic amino acid, and the larger hydrophobic amino acid has a side chain with a molecular weight of at least about 90 g/mol, or at least about 130 g/mol, or at least about 141 g/mol. In other embodiments, the size of the amino acid is measured in terms of the SASA of the hydrophobic side chain, and the larger hydrophobic amino acid has a side chain with a SASA greater than that of alanine, or greater than that of glycine. In other embodiments, AA_{H1} has a hydrophobic side chain with a SASA greater than or equal to about piperidine-2-carboxylate, greater than or equal to about tryptophan, greater than or equal to about phenylalanine, or equal to or greater than about naphthylalanine. In some embodiments, AA_{H1} and AA_{H2} independently have a side with a SASA in the range of from about 200 Å² to about 1000 Å², e.g, about 250 Å², 300 Å², 350 Å², 400 Å², 450 Å², 500 Å², 550 Å², 650 Å², 700 Å², 750 Å², 800 Å², 850 Å², 900 Å², and about 950 Å², inclusive of all values and subranges therebetween.

In some embodiments, AA_{H1} has a side chain side with a SASA of at least about 200 Å², at least about 210 Å2, at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, AA_{H2} has a side chain side with a SASA of at least about 200 Å², at least about 210 Å2, at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, the side chains of AAH₁ and AA_{H2} have a combined SASA of at least about 350 Å², at least about 360 Å², at least about 370 Å², at least about 380 Å2, at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². In some embodiments, AA_{H2} is a hydrophobic amino acid with a side chain having a SASA that is less than or equal to the SASA of the hydrophobic side chain of AA_{H1}. In some embodiments, the presence of the larger hydrophobic amino acid on the N- or C-terminus of the D-Arg or L-Arg, or a combination thereof, in the CPP improves cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

In some embodiments, the CPP does not include a hydrophobic amino acid on the N- and/or C-terminus of AA_{H2}-AA_{H1}-R-r, AA_{H2}-AA_{H1}-r-R, R-r-AA_{H1}-AA_{H2}, or r-R-AA_{H1}-AA_{H2}. In further embodiments, the CPP does not include a hydrophobic amino acid having a side chain which is larger (as described herein) than at least one of AA_{H1} or AA_{H2}. In further embodiments, the CPP does not include a hydrophobic amino acid with a side chain having a surface area greater than AA_{H1}. For example, in embodiments in which at least one of AA_{H1} or AA_{H2} is phenylalanine, the CPP does not further include a naphthylalanine (although the CPP include at least one hydrophobic amino acid which is smaller than AA_{H1} and AA_{H2}, e.g., leucine). In still other embodiments, the CPP does not further include a naphthylalanine in addition to the hydrophobic amino acids in AA_{H2}-AA_{H1}-R-r, AA_{H2}-AA_{H1}-r-R, R-r-AA_{H1}-AA_{H2}, or r-R-AA_{H1}-AA_{H2}.

As discussed above, the chirality of the amino acids (i.e., D or L amino acids) can be selected to improve cytosolic delivery efficiency of the CPP (and the attached cargo as described below). In some embodiments, the hydrophobic amino acid on the N- or C-terminus of an arginine (e.g., AA_{H1}) has the same or opposite chirality as the adjacent arginine. In some embodiments, AA_{H1} has the opposite chirality as the adjacent arginine. For example, when the arginine is D-arg (i.e., “r”), AA_{H1} is a D-AA_{H1}, and when the arginine is L-Arg (i.e., “R”), AA_{H1} is an L-AA_{H1}. Accordingly, in some embodiments, the CPPs disclosed herein may include at least one of the following motifs: D-AA_{H1}-D-arg, D-arg-D-AA_{H1}, L-AA_{H1}-L-Arg, or L-Arg-LAA_{H1}. In particular embodiments, when arginine is D-arg, AA_{H1} can be D-pip. D-nal, D-trp, D-bta, or D-phe. In another non-limiting example, when arginine is L-Arg, AA_{H1} can be L-Pip, L-Nal, L-Trp, L-Bta, or L-Phe. In some embodiments, the presence of the hydrophobic amino acid having the same chirality as the adjacent arginine improves cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold inclusive of all values and subranges therebetween.

In some embodiments, the CPPs described herein (e.g., the CPPs according to Formula IV-A to IV-P) include three arginines. Accordingly, in some embodiments, the CPPs described herein include one of the following sequences: AA_{H2}-AA_{H1}-R-r-R, AA_{H2}-AA_{H1}-R-r-r, AA_{H2}-AA_{H1}-r-R-R, AA_{H2}-AA_{H1}-r-R-r, R-R-r-AA_{H1}-AA_{H2}, r-R-r-AA_{H1}-AA_{H2}, r-r-R-AA_{H1}-AA_{H2}, or, R-r-R-AA_{H1}-AA_{H2}. In particular embodiments, the CPPs described herein have one of the following sequences AA_{H2}-LAA_{H1}-R-r-R, AA_{H2}-DAA_{H1}-r-R-r, r-R-r-DAA_{H1}-AA_{H2}, or R-r-R-LAA_{H1}-AA_{H2}. In some embodiments, the chirality of AA_{H1} and AA_{H2} can be selected to improve cytosolic uptake efficiency, e.g., as described above, where AA_{H1} has the same chirality as the adjacent arginine, and AA_{H1} and AA_{H2} have the opposite chirality.

In some embodiments, the CPPs described herein (e.g., the CPPs according to Formula IV-A to IV-P) include three hydrophobic amino acids. Accordingly, in some embodiments, the CPPs described herein include one of the following sequences: AA_{H3}-AA_{H2}-AA_{H1}-R-r, AA_{H3}-AA_{H2}-AA_{H1}-R-r, AA_{H3}-AA_{H2}-AA_{H1}-r-R, AA_{H3}-AA_{H2}-AA_{H1}-r-R, R-r-AA_{H1}-AA_{H2}-AA_{H3}, R-r-AA_{H1}-AA_{H2}-AA_{H3}, r-R-AA_{H1}-AA_{H2}-AA_{H3}, or, r-R-AA_{H1}-AA_{H2}-AA_{H3}, wherein AA_{H3} is any hydrophobic amino acid described above, e.g., piperidine-2-carboxylate, naphthylalanine, tryptophan, 3-(3-benzothienyl)-alanine, or phenylalanine. In some embodiments, the chirality of AA_{H1}, AA_{H2}, and AA_{H3} can be selected to improve cytosolic uptake efficiency, e.g., as described above, where AAH₁ has the same chirality as the adjacent arginine, and AA_{H1} and AA_{H2} have the opposite chirality. In other embodiments, the size of AA_{H1}, AA_{H2}, and AA_{H3} can be selected to improve cytosolic uptake efficiency, e.g., as described above, where AA_{H3} has a SAS of less than or equal to AA_{H1} and separately/or AA_{H2}.

In some embodiments, AA_{H1} and AA_{H2} have the same or opposite chirality. In certain embodiments, AA_{H1} and AA_{H2} have the opposite chirality. Accordingly, in some embodiments, the CPPs disclosed herein include at least one of the following sequences: D-AA_{H2}-L-AA_{H1}-R-r; L-AA_{H2}-D-AA_{H1}-r-R; R-r-D-AA_{H1}-L-AA_{H2}; or r-R-L-AA_{H1}-D-AA_{H1}, wherein each of D-AA_{H1} and D-AA_{H2} is a hydrophobic amino acid having a D configuration, and each of L-AA_{H1} and L-AA_{H2} is a hydrophobic amino acid having an L configuration. In some embodiments, each of D-AA_{H1} and D-AA_{H2} is independently selected from the group consisting of D-pip, D-nal, D-trp, D-bta, and D-phe. In particular embodiments, D-AA_{H1} or D-AA_{H2} is D-nal. In other particular embodiments, D-AA_{H1} is D-nal. In some embodiments, each of L-AA_{H1} and L-AA_{H2} is independently selected from the group consisting of L-Pip, L-Nal, L-Trp, L-Bta, and L-Phe. In particular embodiments, L-AA_{H1} or L-AA_{H2} is L-Nal. In other particular embodiments, L-AA_{H1} is L-Nal. In some embodiments, the presence of an AA_{H1} and AA_{H2} having an opposite configuration improves the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

### Cargo Moiety

The cargo moiety can comprise any cargo of interest, for example a linker moiety, a detectable moiety, a therapeutic moiety, a targeting moiety, and the like, or any combination thereof. In some examples, the cargo moiety can comprise one or more additional amino acids (e.g., K, UK, TRV); a linker (e.g., bifunctional linker LC-SMCC); coenzyme A; phosphocoumaryl amino propionic acid (pCAP); 8-amino-3,6-dioxaoctanoic acid (miniPEG); L-2,3-diaminopropionic acid (Dap or J); L-β-naphthylalanine; L-pipecolic acid (Pip); sarcosine; trimesic acid; 7-amino-4-methylcourmarin (Amc); fluorescein isothiocyanate (FITC); L-2-naphthylalanine; norleucine; 2-aminobutyric acid; Rhodamine B (Rho); Dexamethasone (DEX); or combinations thereof.

In some examples the cargo moiety can comprise any of those listed in Table 5, or derivatives or combinations thereof.

TABLE 5
Example cargo moieties

| SEQ ID NO | Abbreviation | Sequence* |
|---|---|---|
| SEQ ID NO: 1 | R₅ | RRRRR |
| SEQ ID NO: 2 | A₅ | AAAAA |
| SEQ ID NO: 3 | F₄ | FFFF |
| SEQ ID NO: 4 | PCP | DE(pCAP)LI |
| SEQ ID NO: 5 | A₇ | AAAAAAA |
| SEQ ID NO: 6 | | RARAR |
| SEQ ID NO: 7 | | DADAD |
| SEQ ID NO: 8 | | DΩUD |
| SEQ ID NO: 9 | | UTRV |
| | | D-pThr-Pip-Nal |

*pCAP, phosphocoumaryl amino propionic acid; Ω, norleucine; U, 2-aminobutyric acid; D-pThr is D-phosphothreonine, Pip is L-piperidine-2-carboxylate.

Detectable Moiety

The detectable moiety can comprise any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, a isotope detectable spin resonance label, a paramagnetic moiety, a chromophore, or any combination thereof. In some embodiments, the label is detectable without the addition of further reagents.

In some embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene, perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetrapheny metrylbenzoporphyrin; Pd (II) octaethylporphyrin ketone; Pt (II) octaethylporphyrin ketone; Pd (II) meso-tetra(pentafluorophenyl)porphyrin; Pt (II) meso-tetra (pentafluorophenyl) porphyrin; Ru (II) tris(4,7-diphenyl-1,10-phenanthroline) (Ru (dpp)₃); Ru (II) tris(1,10-phenanthroline) (Ru(phen)₃), tris(2,2′-bipyridine)rutheniurn (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); indium (III) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitirophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluorescein; eosin; 2′,7′-dichlorofluorescein; 5(6)-carboxy-fluorecsein; carboxynaphthofluorescein; 8-hydroxypyrene-1,3,6-trisulfonic acid; semi-naphthorhodafluor; semi-naphthofluorescein; tris (4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; dioctadecylcycloxacarbocyanine; fluorenylmethyloxycarbonyl chloride; 7-amino-4-methylcourmarin (Amc); green fluorescent protein (GFP); and derivatives or combinations thereof.

In some examples, the detectable moiety can comprise Rhodamine B (Rho), fluorescein isothiocyanate (FITC), 7-amino-4-methylcourmarin (Amc), green fluorescent protein (GFP), or derivatives or combinations thereof.

The detectable moiety can be attached to the cell penetrating peptide moiety at the amino group, the carboxylate group, or the side chain of any of the amino acids of the cell penetrating peptide moiety (e.g., at the amino group, the carboxylate group, or the side chain of any amino acid in the CPP).

Therapeutic Moiety

The disclosed compounds can also comprise a therapeutic moiety. In some examples, the cargo moiety comprises a therapeutic moiety. The detectable moiety can be linked to a therapeutic moiety or the detectable moiety can also serve as the therapeutic moiety. Therapeutic moiety refers to a group that when administered to a subject will reduce one or more symptoms of a disease or disorder.

The therapeutic moiety can comprise a wide variety of drugs, including antagonists, for example enzyme inhibitors, and agonists, for example a transcription factor which results in an increase in the expression of a desirable gene product (although as will be appreciated by those in the art, antagonistic transcription factors can also be used), are all included. In addition, therapeutic moiety includes those agents capable of direct toxicity and/or capable of inducing toxicity towards healthy and/or unhealthy cells in the body. Also, the therapeutic moiety can be capable of inducing and/or priming the immune system against potential pathogens.

The therapeutic moiety can, for example, comprise an anticancer agent, antiviral agent, antimicrobial agent, anti-inflammatory agent, immunosuppressive agent, anesthetics, or any combination thereof.

The therapeutic moiety can comprise an anticancer agent. Example anticancer agents include 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The therapeutic moiety can also comprise a biopharmaceutical such as, for example, an antibody.

In some examples, the therapeutic moiety can comprise an antiviral agent, such as ganciclovir, azidothymidine (AZT), lamivudine (3TC), etc.

In some examples, the therapeutic moiety can comprise an antibacterial agent, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene di salicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin di sodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfi soxazole; sulfi soxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

In some examples, the therapeutic moiety can comprise an anti-inflammatory agent.

In some examples, the therapeutic moiety can comprise dexamethasone (Dex).

In other examples, the therapeutic moiety comprises a therapeutic protein. For example, some people have defects in certain enzymes (e.g., lysosomal storage disease). It is disclosed herein to deliver such enzymes/proteins to human cells by linking to the enzyme/protein to one of the disclosed cell penetrating peptides. The disclosed cell penetrating peptides have been tested with proteins (e.g., GFP, PTP1B, actin, calmodulin, troponin C) and shown to work.

In some examples, the therapeutic moiety comprises a targeting moiety. The targeting moiety can comprise, for example, a sequence of amino acids that can target one or more enzyme domains. In some examples, the targeting moiety can comprise an inhibitor against an enzyme that can play a role in a disease, such as cancer, cystic fibrosis, diabetes, obesity, or combinations thereof. For example, the targeting moiety can comprise any of the sequences listed in Table 6.

TABLE 6
Example targeting moieties

| SEQ ID NO | Abbreviation* | Sequence |
|---|---|---|
| SEQ ID NO: 11 | PΘGΛYR | Pro-Pip-Gly-F₂Pmp-Tyr- |
| SEQ ID NO: 12 | SΘIΛΛR | Ser-Pip-Ile-F₂Pmp-F₂Pmp- |
| SEQ ID NO: 13 | IHIΛIR | Ile-His-Ile-F₂Pmp-Ile- |
| SEQ ID NO: 14 | AaIΛΘR | Ala-(D-Ala)-Ile-F₂Pmp-Pip- |
| SEQ ID NO: 15 | ΣSΛΘvR | Fpa-Ser-Pip-F₂Pmp-(D-Val)- |
| SEQ ID NO: 16 | ΘnPΛAR | Pip-(D-Asn)-Pro-F₂Pmp-Ala- |
| SEQ ID NO: 17 | TΨAΛGR | Tyr-Phg-Ala-F₂Pmp-Gly- |
| SEQ ID NO: 18 | AHIΛaR | Ala-His-Ile-F₂Pmp-(D-Ala)- |
| SEQ ID NO: 19 | GnGΛpR | Gly-(D-Asn)-Gly-F₂Pmp-(D-Pro)- |
| SEQ ID NO: 20 | fQΘΛIR | (D-Phe)-Gln-Pip-F₂Pmp-Ile- |
| SEQ ID NO: 21 | SPGΛHR | Ser-Pro-Gly-F₂Pmp-His- |
| SEQ ID NO: 22 | ΘYIΛHR | Pip-Tyr-Ile-F₂Pmp-His- |
| SEQ ID NO: 23 | SvPΛHR | Ser-(D-Val)-Pro-F₂Pmp-His- |
| SEQ ID NO: 24 | AIPΛnR | Ala-Ile-Pro-F₂Pmp-(D-Asn)- |
| SEQ ID NO: 25 | ΣSIΛQF | Fpa-Ser-Ile-F₂Pmp-Gln- |
| SEQ ID NO: 26 | AaΨΛfR | Ala-(D-Ala)-Phg-F₂Pmp-(D-Phe)- |
| SEQ ID NO: 27 | ntΨΛΨR | (D-Asn)-(D-Thr)-Phg-F₂Pmp-Phg- |
| SEQ ID NO: 28 | IPΨΛΩR | Ile-Pro-Phg-F₂Pmp-Nle- |
| SEQ ID NO: 29 | QΘΣΛΘR | Gln-Pip-Fpa-F₂Pmp-Pip- |
| SEQ ID NO: 30 | nAΣΛGR | (D-Asn)-Ala-Fpa-F₂Pmp-Gly- |
| SEQ ID NO: 31 | ntYΛAR | (D-Asn)-(D-Thr)-Tyr-F₂Pmp-Ala- |
| SEQ ID NO: 32 | eAΨΛvR | (D-Glu)-Ala-Phg-F₂Pmp-(D-Val)- |
| SEQ ID NO: 33 | IvΨAR | Ile-(D-Val)-Phg-F₂Pmp-Ala- |
| SEQ ID NO: 34 | YtΨΛAR | Tyr-(D-Thr)-Phg-F₂Pmp-Ala- |
| SEQ ID NO: 35 | nΘΨΛIR | (D-Asn)-Pip-Phg-F₂Pmp-Ile- |
| SEQ ID NO: 36 | ΘnWΛHR | Pip-(D-Asn)-Trp-F₂Pmp-His- |
| SEQ ID NO: 37 | YΘvΛIR | Tyr-Pip-(D-Val)-F₂Pmp-Ile- |
| SEQ ID NO: 38 | nSAΛGR | (D-Asn)-Ser-(D-Ala)-F₂Pmp-Gly- |
| SEQ ID NO: 39 | tnvΛaR | (D-Thr)-(D-Asn)-(D-Val)-F₂Pmp-(D-Ala)- |
| SEQ ID NO: 40 | ntvΛtR | (D-Asn)-(D-Thr)-(D-Val)-F₂Pmp-(D-Thr)- |
| SEQ ID NO: 41 | SItΛYR | Ser-Ile-(D-Thr)-F₂Pmp-Tyr- |
| SEQ ID NO: 42 | nΣnΛlR | (D-Asn)-Fpa-(D-Asn)-F₂Pmp-(D-Leu)- |
| SEQ ID NO: 43 | YnnΛΩR | Tyr-(D-Asn)-(D-Asn)-F₂Pmp-Nle- |
| SEQ ID NO: 44 | nYnΛGR | (D-Asn)-Tyr-(D-Asn)-F₂Pmp-Gly- |
| SEQ ID NO: 45 | AWnΛAR | Ala-Trp-(D-Asn)-F₂Pmp-Ala- |
| SEQ ID NO: 46 | vtHΛYR | (D-Val)-(D-Thr)-His-F₂Pmp-Tyr- |
| SEQ ID NO: 47 | PΨHΛΘR | Pro-Phg-His-F₂Pmp-Pip- |
| SEQ ID NO: 48 | nΨHΛGR | (D-Asn)-Phg-His-F₂Pmp-Gly- |
| SEQ ID NO: 49 | PAHΛGR | Pro-Ala-His-F₂Pmp-Gly- |
| SEQ ID NO: 50 | AYHΛIR | Ala-Tyr-His-F₂Pmp-Ile- |
| SEQ ID NO: 51 | nΘeΛYR | (D-Asn)-Pip-(D-Glu)-F₂Pmp-Tyr- |
| SEQ ID NO: 52 | vSSΛtR | (D-Val)-Ser-Ser-F₂Pmp-(D-Thr)- |
| SEQ ID NO: 53 | aΞt′ϑΦ′yNK | ((D-Ala)-Sar-(D-pThr)-Pp-Nal-Tyr-Gln)-Lys |
| SEQ ID NO: 54 | Tm(aΞt′ϑΦ′RA)Dap | Tm((D-Ala)-Sar-(D-pThr)-Pp-Nal-Arg-Ala)-Dap |
| SEQ ID NO: 55 | Tm(aΞt′ϑΦ′RAa)Dap | Tm((D-Ala)-Sar-(D-pThr)-Pp-Nal-Arg-Ala-(D- |
| | | Ala))-Dap |
| SEQ ID NO: 56 | Tm(aΞtϑΦRAa)Dap | Tm((D-Ala)-Sar-(D-Thr)-Pp-Nal-Arg-Ala-(D- |
| | | Ala))-Dap |
| SEQ ID NO: 57 | Tm(aΞtaΦ′RAa)Dap | Tm((D-Ala)-Sar-(D-Thr)-(D-Ala)-Nal-Arg-Ala- |

*Fpa, Σ: L-4-fluorophenylalanine; Pip, Θ: L-homoproline; Nle, Ω: L-norleucine; Phg, Ψ L-phenylglycine; F₂Pmp, Λ: L-4-(phosphonodifluoromethyl)phenylalanine; Dap, L-2,3-diaminopropionic acid; Nal, Φ′: L-β-naphthylalanine; Pp, ϑ: L-pipecolic acid; Sar, Ξ: sarcosine; Tm, trimesic acid.

The targeting moiety and cell penetrating peptide moiety can overlap. That is, the residues that form the cell penetrating peptide moiety can also be part of the sequence that forms the targeting moiety, and vice a versa.

The therapeutic moiety can be attached to the cell penetrating peptide moiety at the amino group, the carboxylate group, or the side chain of any of the amino acids of the cell penetrating peptide moiety (e.g., at the amino group, the carboxylate group, or the side chain or any of amino acid of the CPP). In some examples, the therapeutic moiety can be attached to the detectable moiety.

In some examples, the therapeutic moiety can comprise a targeting moiety that can act as an inhibitor against Ras (e.g., K-Ras), PTP1B, Pin1, Grb2 SH2, CAL PDZ, and the like, or combinations thereof.

Ras is a protein that in humans is encoded by the RAS gene. The normal Ras protein performs an essential function in normal tissue signaling, and the mutation of a Ras gene is implicated in the development of many cancers. Ras can act as a molecular on/off switch, once it is turned on Ras recruits and activates proteins necessary for the propagation of growth factor and other receptors' signal. Mutated forms of Ras have been implicated in various cancers, including lung cancer, colon cancer, pancreatic cancer, and various leukemias.

Protein-tyrosine phosphatase 1B (PTP1B) is a prototypical member of the PTP superfamily and plays numerous roles during eukaryotic cell signaling. PTP1B is a negative regulator of the insulin signaling pathway, and is considered a promising potential therapeutic target, in particular for the treatment of type II diabetes. PIP1B has also been implicated in the development of breast cancer.

Pin1 is an enzyme that binds to a subset of proteins and plays a role as a post phosphorylation control in regulating protein function. Pin1 activity can regulate the outcome of proline-directed kinase signaling and consequently can regulate cell proliferation and cell survival. Deregulation of Pin1 can play a role in various diseases. The up-regulation of Pin1 may be implicated in certain cancers, and the down-regulation of Pin1 may be implicated in Alzheimer's disease. Inhibitors of Pin1 can have therapeutic implications for cancer and immune disorders.

Grb2 is an adaptor protein involved in signal transduction and cell communication. The Grb2 protein contains one SH2 domain, which can bind tyrosine phosphorylated sequences. Grb2 is widely expressed and is essential for multiple cellular functions. Inhibition of Grb2 function can impair developmental processes and can block transformation and proliferation of various cell types.

It was recently reported that the activity of cystic fibrosis membrane conductance regulator (CFTR), a chloride ion channel protein mutated in cystic fibrosis (CF) patients, is negatively regulated by CFTR-associated ligand (CAL) through its PDZ domain (CAL-PDZ) (Wolde, M et al. J. Biol. Chem. 2007, 282, 8099). Inhibition of the CFTR/CAL-PDZ interaction was shown to improve the activity of APhe508-CFTR, the most common form of CFTR mutation (Cheng, S H et al. Cell 1990, 63, 827; Kerem, B S et al. Science 1989, 245, 1073), by reducing its proteasome-mediated degradation (Cushing, P R et al. Angew. Chem. Int. Ed. 2010, 49, 9907). Thus, disclosed herein is a method for treating a subject having cystic fibrosis by administering an effective amount of a compound or composition disclosed herein. The compound or composition administered to the subject can comprise a therapeutic moiety that can comprise a targeting moiety that can act as an inhibitor against CAL PDZ. Also, the dcompositions or compositions disclosed herein can be administered with a molecule that corrects the CFTR function.

Also disclosed herein are compositions comprising the compounds described herein.

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Methods of Making

In some embodiments, the CPPs described herein are prepared using a peptide according to any of Formula V-A to V-D:

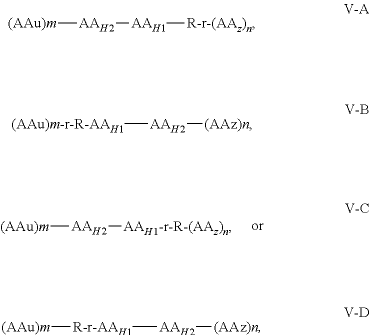

wherein:
each of AA_{H1} and AA_{H2} are independently selected from a hydrophobic amino acid;
at each instance AA_{U} and AA_{Z} are independently selected from an amino acid, with at most one of each AA_{U} and each AA_{Z} being arginine (i.e., with the proviso that the cyclic peptide does not include more than 3 arginines); and
each of m and n are a number from 0 to 6, provided that at least one of m or n is not 0.

In some embodiments, the methods of preparing the peptides disclosed herein comprise contacting a peptide according to any of Formula V-A to V-D (or any amino acid thereof) with a solid support (as described below). In some embodiments, the amino group on the N-terminus and the carboxylate group on the C-terminus of the peptide of Formula V-A, V-B, V-C, or V-D, respectively, independently form a peptide bond. For example, in some embodiments, the N-terminus of the peptide can form a peptide bond with the C-terminus of the peptide, thereby forming a cyclic peptide. In alternative embodiments, the N- and C-terminus can independently form a peptide bond with a cyclization moiety, thereby forming a cyclic peptide. In still other embodiments, the side chain of amino acids in the peptides of Formula V-A, V-B, V-C, or V-D may form a covalent bond, thereby forming a cyclic peptide. In some embodiments, the peptide further comprises a cyclization moiety, which thereby forms a cyclic peptide. The cyclization moiety may be any moiety which is suitable for interacting with amino acids of any of Formula V-A, V-B, V-C, or V-D to thereby form a cyclic peptide. In some embodiments, the cyclization moiety independently forms a peptide bond with an amino group on the N-terminus and a carboxylate group on the C-terminus of amino acids on opposing ends of the peptide of any of Formula V-A, V-B, V-C, or V-D. In embodiments, the cyclization moiety can be a natural or non-natural amino acid. In some embodiments, the one or more amino acids in the CPP can also be part of the cyclization moiety. In some embodiments, the cyclization moeity is a glutamine.

In some embodiments, the peptide further comprises a cargo moiety Xₙ, wherein at least one atom or bond is replaced by a bond to Xₙ, and wherein Xₙ comprises a therapeutic moiety, a targeting moiety, a detectable moiety, or combinations thereof, e.g., as described above.

In some embodiments, the total number of amino acids (including r, R, AA_{H1}, AA_{H2}), in the CPPs of Formula V-A to V-D are in the range of 6 to 10. In some embodiments, the total number of amino acids is 6. In some embodiments, the total number of amino acids is 7. In some embodiments, the total number of amino acids is 8. In some embodiments, the total number of amino acids is 9. In some embodiments, the total number of amino acids is 10.

In some embodiments, the sum of m and n is from 2 to 6. In some embodiments, the sum of m and n is 2. In some embodiments, the sum of m and n is 3. In some embodiments, the sum of m and n is 4. In some embodiments, the sum of m and n is 5. In some embodiments, the sum of m and n is 6. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

As discussed above, the hydrophobicity of amino acids in the CPPs disclosed herein can be selected to improve uptake efficiency. In some embodiments, each of AA_{H1} and AA_{H2} are independently a hydrophobic amino acid having a hydrophobicity value which is greater than that of glycine. In other embodiments, each of AA_{H1} and AA_{H2} are independently a hydrophobic amino acid having a hydrophobicity value which is greater than that of alanine. In still other embodiments, each of AA_{H1} and AA_{H2} are independently an hydrophobic amino acid having a hydrophobicity value which is greater than that of phenylalanine, e.g., as measured using the hydrophobicity scales described above, including Eisenberg and Weiss (Proc. Natl. Acad. Sci. U.S.A 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), (see Table 1 above). In particular embodiments, hydrophobicity is measured using the hydrophobicity scale reported in Engleman, et al.

As described above, the presence of a hydrophobic amino acid on the N- or C-terminus of a D-Arg or L-Arg, or a combination thereof, has also been found to improve the cytosolic uptake of the CPP (and the attached cargo). For example, in some embodiments, the CPPs disclosed herein may include AA_{H1}-D-Arg or D-Arg-AA_{H1}. In other embodiments, the CPPs disclosed herein may include AA_{H1}-L-Arg or L-Arg-AA_{H1}.

As discussed above, the size of the hydrophobic amino acid on the N- or C-terminus of the D-Arg or an L-Arg, or a combination thereof (i.e., AA_{H1}), may be selected to improve cytosolic delivery efficiency of the CPP. For example, a larger hydrophobic amino acid on the N- or C-terminus of a D-Arg or L-Arg, or a combination thereof, improves cytosolic delivery efficiency compared to an otherwise identical sequence having a smaller hydrophobic amino acid. As discussed above, the size of the hydrophobic amino acid can be measured in terms of molecular weight of the hydrophobic amino acid, the steric effects of the hydrophobic amino acid, the solvent-accessible surface area (SASA) of the side chain, or combinations thereof. In some embodiments, the size of the hydrophobic amino acid is measured in terms of the molecular weight of the hydrophobic amino acid, and the larger hydrophobic amino acid has a side chain with a molecular weight of at least about 90 g/mol, or at least about 130 g/mol, or at least about 141 g/mol. In other embodiments, the size of the amino acid is measured in terms of the SASA of the hydrophobic side chain, and the larger hydrophobic amino acid has a side chain with a SASA greater than alanine, or greater than glycine. In other embodiments, AA_{H1} has a hydrophobic side chain with a SASA greater than or equal to about piperidine-2-carboxylate, greater than or equal to about tryptophan, greater than or equal to about phenylalanine, or equal to or greater than about naphthylalanine. In some embodiments, AA_{H1} and AA_{H2} independently have a side with a SASA in the range of from about 200 Å² to about 1000 Å², e.g, about 250 Å², 300 Å², 350 Å², 400 Å², 450 Å², 500 Å², 550 Å², 650 Å², 700 Å², 750 Å², 800 Å², 850 Å², 900 Å², and about 950 Å², inclusive of all values and subranges therebetween.

In some embodiments, AA_{H1} has a side chain side with a SASA of at least about 200 Å², at least about 210 Å2, at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, AA_{H2} has a side chain side with a SASA of at least about 200 Å², at least about 210 Å2, at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, the side chains of AA_{H1} and AA_{H2} have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². In some embodiments, AA_{H2} is a hydrophobic amino acid with a side chain having a SASA that is less than or equal to the SASA of the hydrophobic side chain of AA_{H1}.

In some embodiments, the CPP does not include a hydrophobic amino acid on the N- and/or C-terminus of AA_{H2}-AA_{H1}-R-r, AA_{H2}-AA_{H1}-r-R, R-r-AA_{H1}-AA_{H2}, or r-R-AA_{H1}-AA_{H2}. In alternative embodiments, the CPP does not include a hydrophobic amino acid having a side chain which is larger (as described herein) than at least one of AA_{H1} or AA_{H2}. In further embodiments, the CPP does not include a hydrophobic amino acid with a side chain having a surface area greater than AA_{H1}. For example, in embodiments in which at least one of AA_{H1} or AA_{H2} is phenylalanine, the CPP does not further include a naphthylalanine (although the CPP include at least one hydrophobic amino acid which is smaller than AA_{H1} and AA_{H2}, e.g., leucine). In still other embodiments, the CPP does not include a naphthylalanine in addition to the hydrophobic amino acids in AA_{H2}-AA_{H1}-R-r, AA_{H2}-AA_{H1}-r-R, R-r-AA_{H1}-AA_{H2}, or r-R-AA_{H1}-AA_{H2}.

As discussed above, the chirality of the amino acids (i.e., D or L amino acids) can be selected to improve cytosolic delivery efficiency of the CPP (and the attached cargo as described below). In some embodiments, the hydrophobic amino acid on the N- or C-terminus of an arginine (e.g., AA_{H1}) has the same or opposite chirality as the adjacent arginine. In some embodiments, AA_{H1} has the opposite chirality as the adjacent arginine. For example, when the arginine is D-arg (i.e., “r”), AA_{H1} is a D-AA_{H1}, and when the arginine is L-Arg (i.e., “R”), AA_{H1} is an L-AA_{H1}. Accordingly, in some embodiments, the CPPs disclosed herein may include at least one of the following motifs: D-AA_{H1}-D-arg, D-arg-D-AA_{H1}, L-AA_{H1}-L-Arg, or L-Arg-LAA_{H1}. In particular embodiments, when arginine is D-arg, AA_{H1} can be D-pip, D-nal, D-trp, D-bta, or D-phe. In another non-limiting example, when arginine is L-Arg, AA_{H1} can be L-Pip, L-Nal, L-Trp, L-Bta, or L-Phe.

In some embodiments, the CPPs described herein (e.g., the CPPs according to Formula V-A to V-D) include three arginines. Accordingly, in some embodiments, the CPPs described herein include one of the following sequences: AA_{H2}-AA_{H1}-R-r-R, AA_{H2}-AA_{H1}-R-r-r, AA_{H2}-AA_{H1}-r-R-R, AA_{H2}-AA_{H1}-r-R-r, R-R-r-AA_{H1}-AA_{H2}, r-R-r-AA_{H1}-AA_{H2}, r-r-R-AA_{H1}-AA_{H2}, or, R-r-R-AA_{H1}-AA_{H2}. In particular embodiments, the CPPs described herein have one of the following sequences AA_{H2}-AA_{H1}-R-r-R, AA_{H2}-AA_{H1}-r-R-r, r-R-r-AA_{H1}-AA_{H2}, or R-r-R-AA_{H1}-AA_{H2}. In some embodiments, the chirality of AA_{H1} and AA_{H2} can be selected to improve cytosolic uptake efficiency, e.g., as described above, where AA_{H1} has the same chirality as the adjacent arginine, and AA_{H1} and AA_{H2} have the opposite chirality.

In some embodiments, the CPPs described herein (e.g., the CPPs according to Formula V-A to V-D) include three hydrophobic amino acids. Accordingly, in some embodiments, the CPPs described herein include one of the following sequences: AA_{H3}-AA_{H2}-AA_{H1}-R-r, AA_{H3}-AA_{H2}-AA_{H1}-R-r, AA_{H3}-AA_{H2}-AA_{H1}-r-R, AA_{H3}-AA_{H2}-AA_{H1}-r-R, R-r-AA_{H1}-AA_{H2}-AA_{H3}, R-r-AA_{H1}-AA_{H2}-AA_{H3}, r-R-AA_{H1}-AA_{H2}-AA_{H3}, or, r-R-AA_{H1}-AA_{H2}-AA_{H3}, wherein AA_{H3} is any hydrophobic amino acid described above, e.g., piperidine-2-carboxylate, naphthyl alanine, tryptophan, 3-(3-benzothienyl)-alanine, or phenylalanine. In some embodiments, the chirality of AA_{H1}, AA_{H2}, and AA_{H3} can be selected to improve cytosolic uptake efficiency, e.g., as described above, where AA_{H1} has the same chirality as the adjacent arginine, and AA_{H1} and AA_{H2} have the opposite chirality. In other embodiments, the size of AA_{H1}, AA_{H2}, and AA_{H3} can be selected to improve cytosolic uptake efficiency, e.g., as described above, where AA_{H3} has a SAS of less than or equal to AA_{H1} and separately/or AA_{H2}.

In some embodiments, AA_{H1} and AA_{H2} have the same or opposite chirality. In certain embodiments, AA_{H1} and AA_{H2} have the opposite chirality. Accordingly, in some embodiments, the CPPs disclosed herein include at least one of the following sequences: D-AA_{H2}-L-AA_{H1}-R-r; L-AA_{H2}-D-AA_{H1}-r-R; R-r-D-AA_{H1}-L-AA_{H2}; or r-R-L-AA_{H1}-D-AA_{H1}, wherein each of D-AA_{H1} and D-AA_{H2} is a hydrophobic amino acid having a D configuration, and each of L-AA_{H1} and L-AA_{H2} is a hydrophobic amino acid having an L configuration. In some embodiments, each of D-AA_{H1} and D-AA_{H2} is independently selected from the group consisting of D-pip, D-nal, D-trp, D-bta, and D-phe. In particular embodiments, D-AA_{H1} or D-AA_{H2} is D-nal. In other particular embodiments, D-AA_{H1} is D-nal. In some embodiments, each of L-AA_{H1} and L-AA_{H2} is independently selected from the group consisting of L-Pip, L-Nal, L-Trp, L-Bta, and L-Phe. In particular embodiments, each of L-AA_{H1} and L-AA_{H2} is L-Nal. In other particular embodiments, L-AA_{H1} is L-Nal.

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), Sigma (St. Louis, Mo.), Pfizer (New York, N.Y.), GlaxoSmithKline (Raleigh, N.C.), Merck (Whitehouse Station, N.J.), Johnson & Johnson (New Brunswick, N.J.), Aventis (Bridgewater, N.J.), AstraZeneca (Wilmington, Del.), Novartis (Basel, Switzerland), Wyeth (Madison, N.J.), Bristol-Myers-Squibb (New York, N.Y.), Roche (Basel, Switzerland), Lilly (Indianapolis, Ind.), Abbott (Abbott Park, Ill.), Schering Plough (Kenilworth, N.J.), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenyl sulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for aspartic acid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

### Methods of Use

Also provided herein are methods of use of the compounds or compositions described herein. Also provided herein are methods for treating a disease or pathology in a subject in need thereof comprising administering to the subject an effective amount of any of the compounds or compositions described herein.

Also provided herein are methods of treating cancer in a subject. The methods include administering to a subject an effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

The methods of treatment or prevention of cancer described herein can further include treatment with one or more additional agents (e.g., an anti-cancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The additional anti-cancer agent can also include biopharmaceuticals such as, for example, antibodies.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of killing a tumor cell in a subject. The method includes contacting the tumor cell with an effective amount of a compound or composition as described herein, and optionally includes the step of irradiating the tumor cell with an effective amount of ionizing radiation. Additionally, methods of radiotherapy of tumors are provided herein. The methods include contacting the tumor cell with an effective amount of a compound or composition as described herein, and irradiating the tumor with an effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. An effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection. Prophylactic administration can be used, for example, in the chemopreventative treatment of subjects presenting precancerous lesions, those diagnosed with early stage malignancies, and for subgroups with susceptibilities (e.g., family, racial, and/or occupational) to particular cancers. Therapeutic treatment involves administering to a subject a therapeutically effective amount of the compounds and compositions or pharmaceutically acceptable salts thereof as described herein after cancer is diagnosed.

In some examples of the methods of treating of treating cancer or a tumor in a subject, the compound or composition administered to the subject can comprise a therapeutic moiety that can comprise a targeting moiety that can act as an inhibitor against Ras (e.g., K-Ras), PTP1B, Pin1, Grb2 SH2, or combinations thereof.

The disclosed subject matter also concerns methods for treating a subject having a metabolic disorder or condition. In one embodiment, an effective amount of one or more compounds or compositions disclosed herein is administered to a subject having a metabolic disorder and who is in need of treatment thereof. In some examples, the metabolic disorder can comprise type II diabetes. In some examples of the methods of treating of treating the metabolic disorder in a subject, the compound or composition administered to the subject can comprise a therapeutic moiety that can comprise a targeting moiety that can act as an inhibitor against PTP1B. In one particular example of this method the subject is obese and the method comprises treating the subject for obesity by administering a composition as disclosed herein.

The disclosed subject matter also concerns methods for treating a subject having an immune disorder or condition. In one embodiment, an effective amount of one or more compounds or compositions disclosed herein is administered to a subject having an immune disorder and who is in need of treatment thereof. In some examples of the methods of treating of treating the immune disorder in a subject, the compound or composition administered to the subject can comprise a therapeutic moiety that can comprise a targeting moiety that can act as an inhibitor against Pin1.

The disclosed subject matter also concerns methods for treating a subject having an inflammatory disorder or condition. In one embodiment, an effective amount of one or more compounds or compositions disclosed herein is administered to a subject having an inflammatory disorder and who is in need of treatment thereof.

The disclosed subject matter also concerns methods for treating a subject having cystic fibrosis. In one embodiment, an effective amount of one or more compounds or compositions disclosed herein is administered to a subject having cystic fibrosis and who is in need of treatment thereof. In some examples of the methods of treating the cystic fibrosis in a subject, the compound or composition administered to the subject can comprise a therapeutic moiety that can comprise a targeting moiety that can act as an inhibitor against CAL PDZ.

In some embodiments, the CPPs disclosed herein can be used for detecting or diagnosing a disease or condition in a subject. For example, a CPP can comprise a targeting moiety and/or a detectable moiety that can interact with a target, e.g., a tumor.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane: sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The disclosed compositions are bioavailable and can be delivered orally. Oral compositions can be tablets, troches, pills, capsules, and the like, and can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### EXAMPLES

### Example 1. CPP Synthesis

Peptides were synthesized on Rink amide resin LS (0.2 mmol/g) using standard Fmoc chemistry. The typical coupling reaction contained 5 equiv. of an Fmoc-amino acid, 5 equiv. of 2-(7-aza-1H-benzotriazole-1-yl)-1,1,3,3-tetramethyluronium hexafluorophosphate (HATU) and 10 equiv. of diisopropylethylamine (DIPEA) and was allowed to proceed with mixing for 75 min. After the addition of the last (N-terminal) residue, the allyl group on the C-terminal Glu residue was removed by treatment with Pd(PPh₃)₄, phenylsilane (0.1 and 10 equiv, respectively) in anhydrous DCM (3×15 min). The N-terminal Fmoc group was removed by treatment with 20% piperidine in DMF and the peptide was cyclized by treatment with benzotriazole-1-yl-oxy-tris-pyrrolidino-phosphonium hexafluorophosphate (PyBOP)/HOBt/DIPEA (5, 5, and 10 equiv) in DMF for 3 h. The peptides were deprotected and released from the resin by treatment with 82.5:5:5:5:2.5 (v/v) TFA/thioanisole/water/phenol/ethanedithiol for 2 h. The peptides were triturated with cold ethyl ether (3×) and purified by reversed-phase HPLC on a C₁₈ column. The purity of product (>98%) was assessed by reversed-phase HPLC equipped with an analytical C₁₈ column. The authenticity of each peptide was confirmed by MALDI-TOF mass spectrometry.

To generate fluorescently labelled peptides, an N^{e}-4-methoxytrityl-L-lysine was added to the C-terminus prior to peptide synthesis. After the solid-phase synthesis was complete but before cleavage, the lysine side chain was selectively deprotected using 1% (v/v) TFA in DCM. The resin was incubated with 5 equiv. of a reactive fluorescent labelling reagent (fluorescein isothiocyanate, Lissamine rhodamine B sulfonyl chloride, or naphthofluorescein succinimidyl ester) and 5 equiv. of DIPEA in DMF overnight. The labeled peptide was deprotected, triturated, purified, and analyzed by MALDI-TOF MS as described above.

### Example 2. Cellular Uptake Efficiency

To determine cellular uptake efficiency of the CPPs disclosed herein, HeLa cells were cultured in 12-well plates (1.5×10⁵ cells per well) overnight. The cells were incubated for 2 h with 5 mM naphthofluorescein (NF)-labelled peptide in cellular media. At the end of incubation, the cells were washed with DPBS twice, detached from the plate with 0.25% trypsin, diluted into clear DMEM, pelleted at 250 g for 5 min, washed twice with DPBS, resuspended in DPBS, and analyzed on a BD FACS LSR II flow cytometer. For NF-labelled peptides, a 633-nm laser was used for excitation and the fluorescence emission was analyzed in the APC channel. Absolute cellular uptake efficiency was determined by comparing the concentration (via fluorescence intensity) of the CPP in the cytosol to the concentration in the extracellular medium. Relative cellular uptake efficiency was determined by comparing the cystolic concentration of the CPP to that cystosolic concentration of the control CPP cyclo(FΦRRRRQ) (SEQ ID NO:10).

A number of embodiments of the invention have been described. Nevertheless, it will be understood that various modifications may be made without departing from the spirit and scope of the invention. Accordingly, other embodiments are within the scope of the following claims.

1. A cyclic peptide according to one of Formula I-A to I-E: 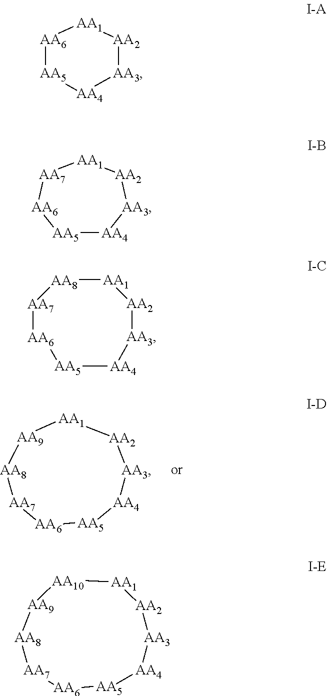
wherein each of AA₁, AA₂, AA₃, AA₄, AA₅, AA₆, AA₇, AA₈, AA₉, and AA₁₀, when present, are independently selected from an amino acid; and
wherein:
two or three of AA₁, AA₂, AA₃, AA₄, AA₅, AA₆, AA₇, AA₈, AA₉, or AA₁₀, when present, are arginine, with the remaining amino acids thereof being an amino acid other than arginine; and
at least two of AA₁, AA₂, AA₃, AA₄, AA₅, AA₆, AA₇, AA₈, AA₉, or AA₁₀, when present, are independently a hydrophobic amino acid.

2. The cyclic peptide of claim 1, wherein each hydrophobic amino acid is independently selected from the group consisting of phenylglycine, leucine, isoleucine, noroleucine, methionine, phenylalanine, homophenylalanine, cyclohexylalanine, tyrosine, piperidine-2-carboxylate, tryptophan, proline, 3-(3-benzothienyl)-alanine, and naphthyl alanine, each of which is optionally substituted with one or more substituents.

3. The cyclic peptide of claim 1, wherein each hydrophobic amino acid is independently piperidine-2-carboxylate, naphthylalanine, tryptophan, 3-(3-benzothienyl)-alanine, or phenylalanine, each of which is optionally substituted with one or more substituents.

4. The cyclic peptide of claim 1, wherein at least one hydrophobic amino acid has a hydrophobicity greater than or equal to phenylalanine.

5. The cyclic peptide of claim 1, wherein at least two of the amino acids have the opposite chirality.

6. The cyclic peptide of any of claims 1-5, wherein at least two of the amino acids have the same chirality.

7. The cyclic peptide of any of claims 1-6, wherein one arginine is adjacent to one hydrophobic amino acid.

8. The cyclic peptide of claim 7, wherein the arginine has the same chirality as the adjacent hydrophobic amino acid.

9. The cyclic peptide of any of claims 1-8, wherein at least two arginines are adjacent to each other.

10. The cyclic peptide of any of claims 1-9, wherein three arginines are adjacent to each other.

11. The cyclic peptide of any of claims 1-10, wherein at least two hydrophobic amino acids are adjacent to each other.

12. The cyclic peptide of any of claims 1-11, wherein at least three hydrophobic amino acids are adjacent to each other.

13. The cyclic peptide of any of claims 1-12, wherein any four adjacent amino acids are AA_{H2}-AA_{H1}-R-r, AA_{H2}-AA_{H1}-r-R, R-r-AA_{H1}-AA_{H2}, or r-R-AA_{H1}-AA_{H2}, wherein each of AA_{H1} and AA_{H2} are independently a hydrophobic amino acid.

14. A cyclic peptide having a structure according to any of Formula II-A to II-D: 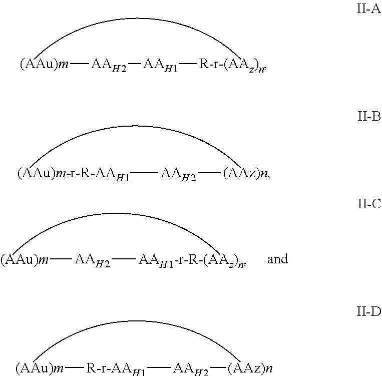
wherein:
each of AA_{H1} and AA_{H2} are independently a hydrophobic amino acid;
at each instance AA_{U} and AA_{Z} are independently any amino acid;
with at most one of each AA_{U} and each AA_{Z} being arginine; and
wherein:
each of m and n are independently a number from 0 to 6, provided that at least one of m or n is not 0 and the total number of amino acids is from 6 to 10.

15. The cyclic peptide of claim 13 or 14, wherein each of AA_{H1} and AA_{H2} are independently piperidine-2-carboxylate, naphthylalanine, tryptophan, 3-(3-benzothienyl)-alanine, or phenylalanine, each of which is optionally substituted with one or more substituents.

16. The cyclic peptide of any of claims 13-15, wherein each of AA_{H1} and AA_{H2} are independently a hydrophobic amino acid having a hydrophobicity greater than or equal to phenylalanine.

17. The cyclic peptide of any of claims 13-16, wherein AA_{H1} is naphthylalanine.

18. The cyclic peptide of any of claims 13-17, wherein AA_{H1} and AA_{H2} are naphthylalanine.

19. The cyclic peptide of any of claims 13-18, wherein AA_{H1} is a hydrophobic amino acid with a side chain having a large solvent-accessible surface area (SASA).

20. The cyclic peptide of claim 19, wherein the large SASA is at least about 200 Å².

21. The cyclic peptide of any of claims 13-20, wherein AA_{H2} is a hydrophobic amino acid with a side chain having a SASA that is less than or equal to the SASA of the side chain of AA_{H1}.

22. The cyclic peptide of any of claims 13-21, wherein when any AA_{U} or any AA_{Z} is a hydrophobic amino acid, said hydrophobic amino acid has a side chain with SASA which is less than AA_{H1}.

23. The cyclic peptide of any of claims 13-22, wherein AA_{H1} and AA_{H2} have the same or opposite chirality.

24. The cyclic peptide of claim 23, wherein AA_{H1} and AA_{H2} have the opposite chirality.

25. The cyclic peptide of any of claims 13-24, wherein AA_{H1} has the same chirality as the adjacent arginine.

26. The cyclic peptide of any of claims 13-25, having one of the following sequences: AA_{H2}-AA_{H1}-R-r-R, AA_{H2}-AA_{H1}-R-r-r, AA_{H2}-AA_{H1}-r-R-R, AA_{H2}-AA_{H1}-r-R-r, R-R-r-AA_{H1}-AA_{H2}, r-R-r-AA_{H1}-AA_{H2}, r-r-R-AA_{H1}-AA_{H2}, or R-r-R-AA_{H1}-AA_{H2}.

27. The cyclic peptide of claim 13-26, having one of the following sequences: AA_{H2}-LAA_{H1}-R-r-R, AA_{H2}-DAA_{H1}-r-R-r, r-R-r-DAA_{H1}-AA_{H2}, or R-r-R-LAA_{H1}-AA_{H2}.

28. The cyclic peptide of any of claims 1-27, having a relative cytosolic delivery efficiency which is improved by an amount in the range of from about 50% to about 500% compared to cyclo(FΦRRRRQ) (SEQ ID NO:10).

29. The cyclic peptide of claim 28, wherein the cyclic peptide has a relative cytosolic delivery efficiency which is improved by about 175% to about 250% compared to cyclo(FΦRRRRQ) (SEQ ID NO:10).

30. The cyclic peptide of claim 29, comprising the following sequence: FfΦRrR (SEQ ID NO:131).

31. The cyclic peptide of claim 28, wherein the cyclic peptide has a relative cytosolic delivery efficiency which is improved by about 150% to about 400% compared to cyclo(FΦRRRRQ) (SEQ ID NO:10).

32. The cyclic peptide of claim 31, comprising the following sequence: fFϕrRr (SEQ ID NO:132).

33. The cyclic peptide of claim 32, wherein the cyclic peptide has a cytosolic delivery efficiency which is improved by about 75% to about 275% compared to cyclo(FΦRRRRQ) (SEQ ID NO:10).

34. The cyclic peptide of claim 33, comprising the following sequence: fFfRrR (SEQ ID NO:133).

35. The cyclic peptide of claim 28, wherein the cyclic peptide has a cytosolic delivery efficiency which is improved by about 150% to about 250% compared to cyclo(FΦRRRRQ) (SEQ ID NO:10).

36. The cyclic peptide of claim 35, comprising the following sequence: FfFrRr (SEQ ID NO:134).

37. The cyclic peptide of claim 36, wherein the cyclic peptide has a cytosolic delivery efficiency which is improved by about 200% to about 450% compared to cyclo(FΦRRRRQ) (SEQ ID NO:10).

38. The cyclic peptide of claim 37, comprising the following sequence: fFϕrRr (SEQ ID NO:135).

39. The cyclic peptide of claim 28, wherein the peptide has a cytosolic delivery efficiency which is improved by about 250% to about 450% compared to cyclo(FΦRRRRQ) (SEQ ID NO:10).

40. The cyclic peptide of claim 39, comprising the following sequence: fΦfrRr (SEQ ID NO:136).

41. A cyclic peptide according to Formula III-A to III-D: 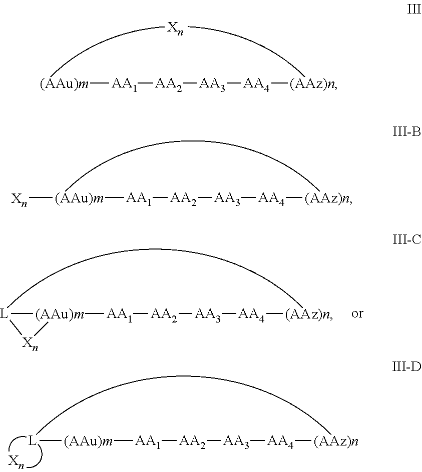
wherein:
each of AA₁, AA₂, AA₃, and AA₄, are independently selected from an amino acid;
at each instance AA_{U} and AA_{Z} are independently selected from an amino acid;
each of m and n are a number from 0 to 6, provided that at least one of m or n is not 0;
Xₙ is a cargo moiety;
L is a linker moiety;
wherein:
two or three of AA₁, AA₂, AA₃, AA₄, each AA_{U}, and each AA_{Z} are arginine, with the remaining amino acids thereof being an amino acid other than arginine;
at least two of AA₁, AA₂, AA₃, AA₄, each AA_{U}, and each AA_{Z} are independently a hydrophobic amino acid; and
wherein when Xₙ is attached to AAᵤ, m is not 0.

42. The cyclic peptide of claim 41, wherein each hydrophobic amino acid is independently selected from the group consisting of phenylglycine, leucine, isoleucine, noroleucine, methionine, phenylalanine, homophenylalanine, cyclohexylalanine, tyrosine, piperidine-2-carboxylate, tryptophan, proline, 3-(3-benzothienyl)-alanine, and naphthyl alanine, each of which is optionally substituted with one or more substituents.

43. The cyclic peptide of claim 41, wherein each hydrophobic amino acid is independently piperidine-2-carboxylate, naphthylalanine, tryptophan, 3-(3-benzothienyl)-alanine, or phenylalanine, each of which is optionally substituted with one or more substituents.

44. The cyclic peptide of claim 41, wherein at least one hydrophobic amino acid has a hydrophobicity greater than or equal to phenylalanine.

45. The cyclic peptide of claim 41, wherein at least two of the amino acids have the opposite chirality.

46. The cyclic peptide of any of claims 41-45, wherein at least two of the amino acids have the same chirality.

47. The cyclic peptide of any of claims 41-46, wherein one arginine is adjacent to one hydrophobic amino acid.

48. The cyclic peptide of claim 47, wherein the arginine has the same chirality as the adjacent hydrophobic amino acid.

49. The cyclic peptide of any of claims 41-48, wherein at least two arginines are adjacent to each other.

50. The cyclic peptide of any of claims 41-49, wherein three arginines are adjacent to each other.

51. The cyclic peptide of any of claims 41-50, wherein at least two hydrophobic amino acid are adjacent to each other.

52. The cyclic peptide of any of claims 41-51, wherein at least three hydrophobic amino acid are adjacent to each other.

53. The cyclic peptide of any of claims 41-52, wherein any four adjacent amino acids are AA_{H2}-AA_{H1}-R-r, AA_{H2}-AA_{H1}-r-R, R-r-AA_{H1}-AA_{H2}, or r-R-AA_{H1}-AA_{H2}, wherein each of AA_{H1} and AA_{H2} are independently a hydrophobic amino acid.

54. The cyclic peptide of any of claims 41-52, wherein the peptide as a structure according to any of Formula IV-A to IV-P: 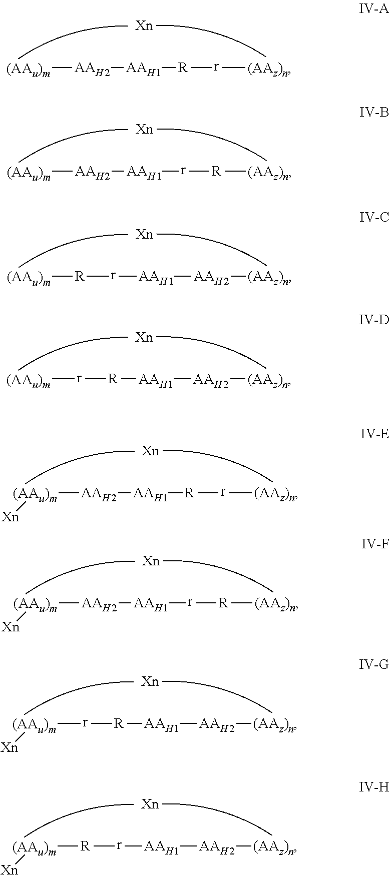 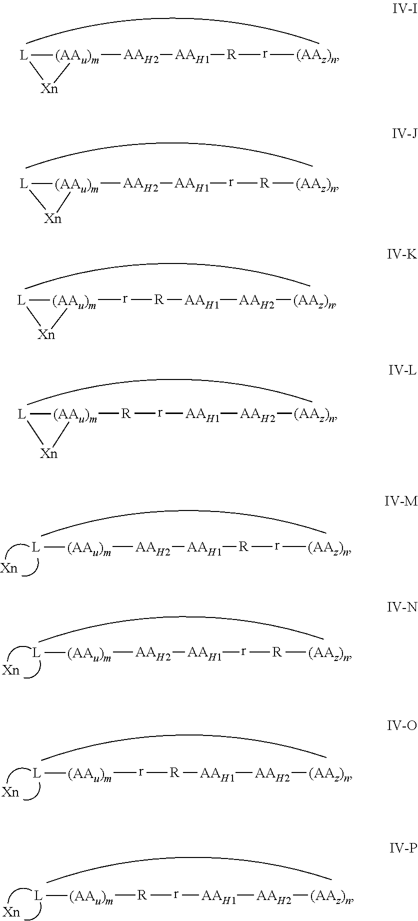
wherein:
each of AA_{H1} and AA_{H2} are independently a hydrophobic amino acid.

55. The cyclic peptide of claim 52 or 54, wherein each of AA_{H1} and AA_{H2} are independently piperidine-2-carboxylate, naphthylalanine, tryptophan, 3-(3-benzothienyl)-alanine, or phenylalanine, each of which is optionally substituted with one or more substituents.

56. The cyclic peptide of any of claims 53-55, wherein each of AA_{H1} and AA_{H2} are independently a hydrophobic amino acid having a hydrophobicity greater than or equal to phenylalanine.

57. The cyclic peptide of any of claims 53-56, wherein AA_{H1} is naphthylalanine.

58. The cyclic peptide of any of claims 53-57, wherein AA_{H} and AA_{H2} are naphthylalanine.

59. The cyclic peptide of any of claims 53-57, wherein AA_{H1} is a hydrophobic amino acid with a side chain having a large solvent-accessible surface area (SASA).

60. The cyclic peptide of claim 59, wherein the large SASA is at least about 200 Å².

61. The cyclic peptide of any of claims 53-50, wherein when any AA_{U} or any AA_{Z} is a hydrophobic amino acid, said hydrophobic amino acid has a side chain with SASA which is less than AA_{H1}.

62. The cyclic peptide of any of claims 53-61, wherein the cyclic peptide does not include a hydrophobic amino acid with a side chain having SASA which is greater than AA_{H1}.

63. The cyclic peptide of any of claims 53-62, wherein AA_{H1} and AA_{H2} have the same or opposite chirality.

64. The cyclic peptide of claim 63, wherein AA_{H1} and AA_{H2} have the opposite chirality.

65. The cyclic peptide of any of claims 53-64, wherein AA_{H1} has the same chirality as the adjacent arginine.

66. The cyclic peptide of any of claims 53-65, having one of the following sequences: AA_{H2}-AA_{H1}-R-r-R, AA_{H2}-AA_{H1}-R-r-r, AA_{H2}-AA_{H1}-r-R-R, AA_{H2}-AA_{H1}-r-R-r, R-R-r-AA_{H1}-AA_{H2}, r-R-r-AA_{H1}-AA_{H2}, r-r-R-AA_{H1}-AA_{H2}, or, R-r-R-AA_{H1}-AA_{H2}.

67. The cyclic peptide of claim 53-66, having one of the following sequences AA_{H2}-LAA_{H1}-R-r-R, AA_{H2}-DAA_{H1}-r-R-r, r-R-r-DAA_{H1}-AA_{H2}, or R-r-R-LAA_{H1}-AA_{H2}.

68. A peptide comprising Formula V-A to V-D: 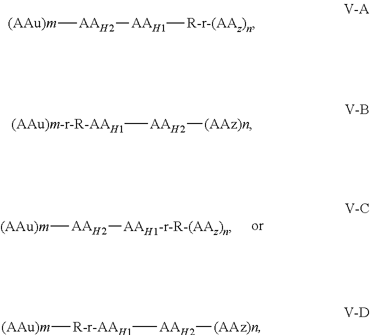
wherein:
each of AA_{H1} and AA_{H2} are independently selected from a hydrophobic amino acid;
at each instance, AA_{U} and AA_{Z}, are independently selected from an amino acid, with at most one of each AA_{U} and each AA_{Z} being arginine, and
each of m and n are a number from 0 to 6, provided that at least one of m or n is not 0.

69. The peptide of claim 68, wherein an amino group on the N-terminus or a carboxylate group on the C-terminus of any of Formula V-A to V-D independently form a peptide bond.

70. The peptide of claim 68, further comprising a cyclization moiety, wherein the cyclization moiety, which thereby forms a cyclic peptide.

71. The peptide of claim 70, wherein the cyclization moiety independently forms a peptide bond with an amino group on the N-terminus or a carboxylate group on the C-terminus of any of Formula V-A to V-D.

72. The peptide of any of claims 68-71, further comprising a cargo moiety (Xₙ), wherein at least one atom or bond is replaced by a bond to Xₙ.

73. The cyclic peptide of any of claims 68-72, wherein each of AA_{H1} and AA_{H2} are independently piperidine-2-carboxylate, naphthyl alanine, tryptophan, 3-(3-benzothienyl)-alanine, or phenylalanine, each of which is optionally substituted with one or more substituents.

74. The cyclic peptide of any of claims 68-73, wherein each of AA_{H1} and AA_{H2} are independently a hydrophobic amino acid having a hydrophobicity greater than or equal to phenylalanine.

75. The cyclic peptide of any of claims 68-74, wherein AA_{H1} is naphthylalanine.

76. The cyclic peptide of any of claims 68-75, wherein AA_{H1} and AA_{H2} are naphthylalanine.

77. The cyclic peptide of any of claims 68-76, wherein AA_{H1} is a hydrophobic amino acid with a side chain having a large solvent-accessible surface area (SASA).

78. The cyclic peptide of claim 77, wherein the large SASA is at least about 200 Å².

79. The cyclic peptide of any of claims 68-78, wherein AA_{H2} is a hydrophobic amino acid with a side chain having a SASA that is less than or equal to the SASA of the hydrophobic side chain of AA_{H1}.

80. The cyclic peptide of any of claims 68-79, wherein when any AA_{U} or any AA_{Z} is a hydrophobic amino acid, said hydrophobic amino acid has a side chain with SASA which is less than AA_{H1}.

81. The cyclic peptide of any of claims 68-80, wherein AA_{H1} and AA_{H2} have the same or opposite chirality.

82. The cyclic peptide of claim 81, wherein AA_{H1} and AA_{H2} have the opposite chirality.

83. The cyclic peptide of any of claims 68-82, wherein AA_{H1} has the same chirality as the adjacent arginine.

84. The cyclic peptide of any of claims 68-83, having one of the following sequences: AA_{H2}-AA_{H1}-R-r-R, AA_{H2}-AA_{H1}-R-r-r, AA_{H2}-AA_{H1}-r-R-R, AA_{H2}-AA_{H1}-r-R-r, R-R-r-AA_{H1}-AA_{H2}, r-R-r-AA_{H1}-AA_{H2}, r-r-R-AA_{H1}-AA_{H2}, or, R-r-R-AA_{H1}-AA_{H2}.

85. The cyclic peptide of claim 68-84, having one of the following sequences AA_{H2}-LAA_{H1}-R-r-R, AA_{H2}-DAA_{H1}-r-R-r, r-R-r-DAA_{H1}-AA_{H2}, or R-r-R-LAA_{H1}-AA_{H2}.

86. A method of treating or diagnosing a patient in need thereof, comprising administering cyclic peptide according to any of claims 41-67.

87. A composition comprising the cyclic peptide according to any of claims 41-67.

88. The peptide of any of claims 68-85, wherein the cytosolic delivery efficiency of the peptide comprising any of Formula V-A to V-D is improved by an amount in the range of from about 50% to about 500% compared to cyclo(FΦRRRRQ) (SEQ ID NO:10).

89. The peptide of claim 88, wherein the peptide comprising any of Formula V-A to V-D comprises the following sequence: FfΦRrR (SEQ ID NO:131).

90. The peptide of claim 88, wherein the peptide comprising any of Formula V-A to V-D comprises the following sequence: fFϕrRr (SEQ ID NO:132).

91. The peptide of claim 88, wherein the peptide comprising any of Formula V-A to V-D comprises the following sequence: fFfRrR (SEQ ID NO:133).

92. The peptide of claim 88, wherein the peptide comprising any of Formula V-A to V-D comprises the following sequence: FfFrRr (SEQ ID NO:134).

93. The peptide of claim 88, wherein the peptide comprising any of Formula V-A to V-D comprises the following sequence: fFϕrRr (SEQ ID NO:135).

94. The peptide of claim 88, wherein the peptide comprising any of Formula V-A to V-D comprises the following sequence: fΦfrRr (SEQ ID NO:136).
